(19) United States
(12) Patent Application Publication
(10) Pub. No.: US 2016/0367705 A1
(43) Pub. Date: Dec. 22, 2016

(54) NANOPARTICLES COMPRISED OF SHELLS ASSOCIATED WITH CHARGED ENTITIES AND FORMED FROM MONOMERS AND METHODS OF MAKING AND USING NANOPARTICLES

(71) Applicant: Marc WALTERS, New Rochelle, NY (US)

(72) Inventor: Marc WALTERS, New Rochelle, NY (US)

(73) Assignee: New York University, New York, NY (US)

(21) Appl. No.: 15/184,822

(22) Filed: Jun. 16, 2016

Related U.S. Application Data
(60) Provisional application No. 62/180,406, filed on Jun. 16, 2015.

(51) Int. Cl. A61K 49/18; A61K 9/50; A61K 9/51

(52) U.S. Cl. CPC A61K 49/1857; A61K 9/5115; A61K 9/5123; A61K 9/5089; A61K 9/5146

(57) ABSTRACT

The present invention relates to relates to a nanoparticle that includes a shell formed from a first monomer having a first charge and a second monomer different than the first monomer. The first and second monomers are copolymerized, and the shell encapsulates a core region and is associated with a charged entity having a second charge of opposite sign to the first charge. The present invention further relates to a nanoparticle having a neutral charge. The present invention further relates to nanoparticle dispersions, methods of making the nanoparticle, methods of method of imaging, methods of delivering drugs, and methods of delivering a high concentration of contrast enhancing and/or imaging agents.

This application claims the priority benefit of U.S. Provisional Patent Application Ser. No. 62/180,406, filed Jun. 16, 2015, which is hereby incorporated by reference in its entirety.

This invention was made with government support under CRIF/CHE-0840277 awarded by the National Science Foundation and DMR-0820341 awarded by the NSF MRSEC Program. The government has certain rights in the invention.

### FIELD OF THE INVENTION

The present application relates to nanoparticles comprised of shells associated with charged entities and formed from monomers and methods of making and using nanoparticles.

### BACKGROUND OF THE INVENTION

Inverse emulsion polymerization is technologically important for the synthesis of high molecular weight, linear water-soluble polymers. Candau et al., “Polymerization in Nanostructured Media: Applications to the Synthesis of Associative Polymers,” Macromol. Symp. 179:13-25 (2002). The lattice instability and product sedimentation that sometimes limits this method is avoided with inverse microemulsion polymerization. Candau et al., “Kinetic-Study of the Polymerization of Acrylamide in Inverse Microemulsion,” J. Polym. Sci. Pol. Chem. 23:193-214 (1985), Voortmans et al., “Structure and Reactivity in Reverse Micelles,” ed. M. P. Pileni (New York: Elsevier) pp. 221-9 (1989). Reverse micelles constitute one class of inverse microemulsions employed for polymerization. Stoffer et al., “Polymerization in Water-in-Oil Micro-Emulsion Systems,” J. Polym. Sci. Pol. Chem. 18:2641-8 (1980). The formation of high molecular weight polymers and copolymers in reverse micelle solutions is a consequence of particle collisions and the transfer of monomers. Candau et al., “Carbon-13 NMR Study of the Sequence Distribution of Poly(acrylamide-co-sodium acrylates) Prepared in Inverse Microemulsions,” Macromolecules 19:1895-902 (1986), Candau et al., “Characterization of Poly(acrylamide-co-acrylates) Obtained by Inverse Microemulsion Polymerization,” Colloid Polym. Sci. 264:676-82 (1986). In most cases the monomers and surfactants are chemically independent, exhibiting no significant affinities.

Crosslinking within water core reverse micelles with the aim of forming organic nanoparticles from one dimensional polymers has been reported. Jung et al., “Synthesis and Characterization of Cross-Linked Reverse Micelles,” J. Am. Chem. Soc. 125:5351-5 (2003); Hammouda et al., “Synthesis of Nanosize Latexes by Reverse Micelle Polymerization,” Langmuir 11:3656-9 (1995); and Voortmans et al., “Polymerization of N,N-Didodecyl-N-Methyl-N-(2-(Methacryloyloxy)Ethyl)Ammonium Chloride, an Inverse Micelle Forming Detergent,” Macromolecules 21:1977-80 (1988).

There is a rich literature on the formation of phases and particles from polysaccharides that contain glucuronic acid (gluc-H), a widely occurring sugar that is secreted by mucus membranes and serves as a component of proteoglycans. Abdel-Mohsen et al., “Green Synthesis of Hyaluronan Fibers With Silver Nanoparticles,” Carbohydr. Polym. 89:411-22 (2012) and Chudobova et al., “Complexes of Silver(I) Ions and Silver Phosphate Nanoparticles with Hyaluronic Acid and/or Chitosan as Promising Antimicrobial Agents for Vascular Grafts,” Int. J. Mol. Sci. 14:13592-614 (2013). It is of recognized technological and biomedical importance, as a major component of the polymer hyaluronic acid that is employed for clinical applications. Allison et al., “Review. Hyaluronan: A Powerful Tissue Engineering Tool,” Tissue Eng. 12:2131-40 (2006). In addition, hyaluronic acid composites embedded with silver ions or silver nanoparticles have been investigated as antibacterial agents for the treatment of wounds. Abdel-Mohsen et al., “Antibacterial Activity and Cell Viability of Hyaluronan Fiber with Silver Nanoparticles,” Carbohydr. Polym. 92:1177-87 (2013); Anisha et al., “Chitosan-Hyaluronic Acid/Nano Silver Composite Sponges for Drug Resistant Bacteria Infected Diabetic Wounds,” Int. J. Biol. Macromol. 62:310-20 (2013); Choi et al., “Studies on Gelatin-Based Sponges. Part III: A Comparative Study of Crosslinked Gelatin/Alginate, Gelatin/Hyaluronate and Chitosan/Hyaluronate Sponges and Their Application as a Wound Dressing in Full-Thickness Skin Defect of Rat,” J. Mater. Sci.: Mater. Med. 12:67-73 (2001); Kemp et al., “Hyaluronan- and Heparin-Reduced Silver Nanoparticles With Antimicrobial Properties,” Nanomedicine (London, U. K.) 4:421-9 (2009); Park et al., “Polysaccharides and Phytochemicals: A Natural Reservoir for the Green Synthesis of Gold and Silver Nanoparticles,” IET Nanobiotechnol. 5:69-78 (2011); and Xia et al., “Green Synthesis of Silver Nanoparticles by Chemical Reduction With Hyaluronan,” Carbohydr. Polym. 86:956-61 (2011). Recent papers describe monomeric glucuronate anions (gluc) as surface ligands for inorganic nanoparticles of Ln₂O₃ to render them dispersible in aqueous media for imaging. Kim et al., “Ligand-Size Dependent Water Proton Relaxivities in Ultrasmall Gadolinium Oxide Nanoparticles and In Vivo T-1 MR Images in a 1.5 T MR field,” PCCP 16:19866-73 (2014); Kattel et al., “Water-Soluble Ultrasmall Eu2O3 Nanoparticles as a Fluorescent Imaging Agent: In Vitro and In Vivo Studies,” Colloid Surface A 394:85-91 (2012); and Kattel et al., “A Facile Synthesis, In Vitro and In Vivo MR Studies of D-Glucuronic Acid-Coated Ultrasmall Ln(2)O(3) (Ln=Eu, Gd, Dy, Ho, and Er) Nanoparticles as a New Potential MRI Contrast Agent,” Acs Appl. Mater. Inter. 3:3325-34 (2011).

The number of novel nanoparticles is large. Among the first nanoparticles to be developed were those constructed with complex organic surface layers on a metal core such as gold or mineral core such as silica. Other nanoparticles have been constructed with a polymeric organic core consisting of micelles, dendrimers, dextran, or PLGA. These nanoparticles either have no core-carrying capacity or a low carrying capacity. In most cases each iteration of particle development has required a high level of sophistication and a relatively large laboratory staff.

The present invention is directed to overcoming these and other deficiencies in the art.

### SUMMARY OF THE INVENTION

One aspect of the present invention relates to a nanoparticle. The nanoparticle includes a shell formed from a first monomer having a first charge and a second monomer different than the first monomer. The first and second monomers are copolymerized, and the shell encapsulates a core region and is associated with a charged entity having a second charge of opposite sign to the first charge.

Another aspect of the present invention relates to a nanoparticle. The nanoparticle includes a shell formed from a first monomer and a second monomer different from the first monomer. The first and second monomer are copolymerized, and the shell encapsulates a core region and has a neutral charge.

Another aspect relates to a dispersion. The dispersion includes a nanoparticle in accordance with the present invention and a medium selected from the group consisting of water, methanol, dimethyl sulfoxide, chloroform, methylene chloride, and mixtures thereof. The nanoparticle is dispersed in the medium.

Another aspect of the present invention relates to a method of making a nanoparticle. The method includes providing a first monomer having a first charge and providing a charged entity having a second charge of opposite sign to the first charge. The first monomer is contacted with the charged entity in an aqueous medium under conditions effective to form a complex where the first monomer is associated with the charged entity. A second monomer is provided and contacted with the complex under conditions effective to form a shell of the first and second monomers which have been copolymerized. The shell encapsulates a core region containing an aqueous medium.

Another aspect of the present invention relates to a method of imaging. The method includes providing the nanoparticle of the present invention where the nanoparticle encapsulates an imaging agent in the core region. A subject to be imaged is provided and the nanoparticle is administered to said subject. An imaging procedure is conducted on said subject to which the nanoparticles have been administered.

Another aspect of the present invention relates to a method of delivering drugs. The method includes providing a nanoparticle of the present invention with a drug encapsulated in the core and a subject to be treated. The nanoparticle is administered to the subject under conditions effective to deliver drugs.

Another aspect of the present invention relates to a method of delivering a high concentration of contrast enhancing and/or imaging agents. The method includes providing a nanoparticle in accordance with the present invention with a contrast enhancing and/or imaging agent encapsulated in the core and providing a subject to be treated. The nanoparticle is then administered to the subject.

A synthetic approach is described in the present application, where monomer reactants are copolymerized to form a shell encapsulating a core region. For example, the reactants can be assembled within a reverse micelle where the anionic monomers are counterions of a quaternary ammonium surfactant. The introduction of a second monomer (e.g., crosslinking agent) in the bulk phase induces polymerization by anion or cation addition, which results in the formation of a nanoparticulate hyperbranched copolymer whose dimensions are dictated by reverse micelles comprised of the anionic monomer and the cationic surfactant. The anion in this system is, for example, the sugar glucuronic acid in the carboxylate form and the crosslinker is, for example, epichlorohydrin. The attractive features of this system are the ready availability of the precursors, the uniformity of the product, and the utility of the nanoparticulate product, that favorably resembles dendrimers. A feature of the present invention is that copolymerization is enabled by the use of a charged entity (e.g., a cationic surfactant) that stabilizes the copolymerized monomers (e.g., reverse micelles) without cosurfactants.

In particular, glucuronic acid (gluc-H) in its carboxylate form (gluc) has been paired with a cationic surfactant, cetyldimethylammonium acetamide (CDA), to assemble uniform reverse micelles in chloroform solution. Carboxylate-rich polyanionic nanoparticles are formed by the reaction of epichlorohydrin with glucuronate (gluc) in reverse micelles. This reaction produces hyperbranched polymeric gluc particles (CDA gluc-NP) that are uniform and average ˜14 nm in diameter as determined by dynamic light scattering (DLS) and transmission electron microscopy (TEM). Silver ions were employed as a particle stain for TEM. Silver ion uptake was accompanied by autoreduction to silver metal, which enabled characterization of the resulting composite by evaluation of the silver surface plasmon resonance spectrum. Preliminary evidence suggests that CDA gluc-NP reacts with preformed silver nanoparticles to form superstructures.

Alcohol co-surfactants are required for reverse micelle formation with common cationic surfactants such cetyltrimethylammonium halide, CTAX. The reverse micelles of the present invention can be loaded with cargo that is useful for biomedical imaging, for photonics, and materials synthesis. After loading, the monomers may be crosslinked to form stable polymeric nanoparticles. The unloaded stable nanoparticles can form building blocks by attaching to, for example, cationic nanoparticles or metal nanoparticles, and can thereby self-assemble into colloidal crystals and related structures.

The shell can incorporate generic crosslinker molecules and sugar acids to give products that are soluble in either organic or aqueous media.

A hallmark of the present invention is simplicity and flexibility providing particles that are stable, small, uniform, and soluble in either organic or aqueous solution. Ease of fabrication is an element in constraining cost. The product is designed to incorporate a rich variety of water-soluble compounds that are either wholly organic or can include metal complexes.

### BRIEF DESCRIPTION OF THE DRAWINGS

FIG. 1 shows an electron TEM image of composite gluc-NP@Ag at a magnification of 50000×. The reference bar is 20 nm.

FIG. 2 illustrates a powder X-Ray Powder Diffraction (“XRD”) pattern for CDA gluc NP Ag, reflectance data.

FIG. 3 shows a TGA profile for the reaction product of CDAgluc-NP with AgNO₃ in water.

FIGS. 4A-B depict superstructures derived from the reaction of CDA gluc-NP with Ag@myr in chloroform. FIG. 4A shows SEM images microrods. FIG. 4B shows clusters.

FIG. 5 is an exemplary scheme of forming a hyperbranched polymer nanoparticle or nanocapsule.

FIG. 6 shows Atomic Force Microscopy (AFM) images for CDAGluc-NP.

FIG. 7 illustrates a synthetic overview for manganese oxide containing nanoparticles.

FIG. 8 shows Dynamic Light Scattering Data (DLS) for the manganese oxide nanostructures.

FIG. 9 shows low magnification TEM image for manganese oxide nanostructure.

FIG. 10 shows high magnification TEM image for manganese oxide nanostructure.

FIG. 11 shows details from the single crystal X-ray structure of dodecyl dimethylammonium acetamido chloride (“DDA-Cl”).

### DETAILED DESCRIPTION OF THE INVENTION

One aspect of the present invention relates to a nanoparticle. The nanoparticle includes a shell formed from a first monomer having a first charge and a second monomer different than the first monomer. The first and second monomers are copolymerized, and the shell encapsulates a core region and is associated with a charged entity having a second charge of opposite sign to the first charge.

The nanoparticle may have a shape that is spherical, rod shaped, polyhedral, cylindrical, or branched cylindrical. In one embodiment, the nanoparticle comprises a polymeric shell that is formally two-dimensional. The nanoparticle may also be a hyperbranched polymeric nanoparticle and/or a reticulated capsule in varying embodiments.

The nanoparticle units are formed by copolymerization of the first and second monomers in the presence or absence of one or more charged entities (i.e., surfactant). In one embodiment, the first charge is negative and the second charge is positive. Alternatively, the first charge may be positive and the second charge may be negative.

In one embodiment, the number of polymeric units in the nanoparticle ranges from 10 to 5000, for instance from 20 to 400, for hyperbranched polymer nanoparticle formed from the polymeric units. In another embodiment, the number of polymeric units ranges from 10,000 to 200,000, for instance from 15,000 to 200,000, for a hyperbranched polymer nanocapsule formed from the hyperbranched polymeric units.

The hyperbranched structures form by copolymerizing (i.e., linking) the first monomer and second monomers. In one embodiment, the linking method is similar to crosslinking copolymer blocks to form micelles and reverse micelles. See, e.g., Read & Armes, “Recent Advances in Shell Cross-Linked Micelles,” Chem. Comm. 29:3021-25 (2007), which is hereby incorporated by reference in its entirety.

In one embodiment, the first monomer is a deprotonated sugar acid comprising three or more hydroxyl groups and may further include one or two carboxylate groups. Linking of the hydroxyl groups is enabled by the use of a charged entity (e.g., a cationic surfactant) that assembles a closely spaced array of the monomers without cosurfactants. In another embodiment, the first monomer is selected from the group consisting of carboxylic acids, amines, alcohols, thiols, aldehydes, ketones, ethers, esters, nitriles, imides, or any salt thereof. In another embodiment, the first monomer is an anionic monomer and may be a sugar acid selected from the group consisting of aldonic acids, ulosonic acids, uronic acids, and aldaric acids. For example, the sugar acid may be the uronic acid glucuronic acid.

Suitable second monomers include, but are not limited to, epichlorohydrin, divinyl sulfone (DVS), citric acid, diacid chloride, diepoxybutane, diepoxyoctane, butanediol-diglycidyl ether (BDDE), ethylene glycol diglycidyl ether, polyglycerol polyglycidyl ether, ethylene sulfide, glutaraldehyde, bromoacetic anhydride, acrylic anhydride, 3-mercaptopropanoate, thioacetic acid, divinyladipate (DVA), POCl₃, sodium trimetaphosphate, diethylenetriaminepentaacetic acid (DTPA), cystine, DTPA bisanhydride, 1,3,5-triazine-2,4,6-triamine hexaacetic acid (H6TTHA), H6TTHA tris anhydride, and mixtures thereof. Additional descriptions for modifications of second monomers can be found in Schanté et al., “Chemical Modifications of Hyaluronic Acid for the Synthesis of Derivatives for a Broad Range of Biomedical Applications,” Carbohydrate Polymers 85:469-89 (2011), which is hereby incorporated by reference in its entirety.

Methods of modifying substituents and copolymerization reactions between the first and second monomers to form a nanoparticle are those known to one skilled in the art. For instance, copolymerization can occur through ether or ester, in the presence of a terminal —OH group, or disulfide bond formations where there is a terminal —SH or—S. group. For example, copolymerization can occur through the formation of ether, ester, amide, thioether, or disulfide bonds between the monomers. For example, copolymerization can occur through the reaction of an amine on one of the monomers and an anhydride on another monomer. Copolymerization can also occur through the reaction of an amine on one of the monomers and a carboxylic acid or carboxylate group on another monomer. Such bonds are formed through a reaction between a functional group on one of the monomers with another functional group of the other monomer. These functional groups are well known to a person of skill in the art. In a preferred embodiment, copolymerization occurs through the formation of the ether bonds between the monomers. These bonds are formed through a reaction between a hydroxyl group of one of the monomers and an epoxide group of another monomer. Alternatively, ether bonds can be formed by reacting alkenes with alcohol; alcohol with another alcohol; and alcohol or alkoxide with an alkyl halide. Disulfide bonds may be formed through a reaction between a thiol group of one of the monomers and a thiol group of another monomer. Ester bonds in the copolymerization reaction of the present invention can be formed by through a reaction between an alcohol group of one of the monomers with carboxylic acid group of another monomer.

In one embodiment, the first monomer and the second monomer are covalently linked. Exemplary reactions for an example compound CDA-gluc include various linkers to form hyperbranched polymeric units are shown infra, in Scheme 4. The polymer repeat unit of CDA gluc-NP may, in one embodiment, have the formula C₃₂N₂O₁₀H₆₀. The CDA gluc-NP may also have a molecular mass of 632.83 g/mol.

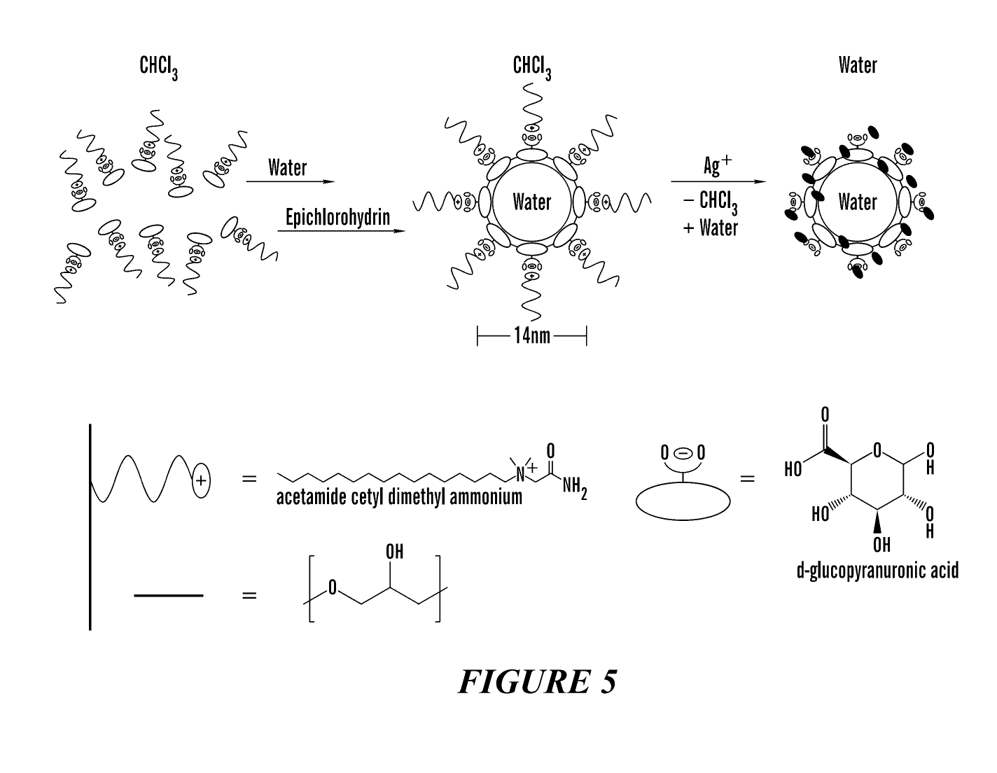

An exemplary scheme of forming a hyperbranched polymer nanoparticle or nanocapsule is shown in FIG. 5. For example, the first monomer may be glucuronic acid (gluc-H) in its carboxylate form (gluc) which can be paired with a charged entity such as a cationic surfactant like, but not limited to, cetyldimethylammonium acetamide (CDA). The pair can assemble uniform reverse micelles in a solvent such as chloroform. This scheme is merely an example of the present invention that is not limited to the use of gluc and CDA exclusively. Rather, gluc could be replaced with any first monomer as described above. Similarly, gluc could be paired with any charged entity as described above.

Carboxylate-rich polyanionic nanoparticles may be formed by the reaction of a second monomer such as epichlorohydrin with the first monomer (e.g., gluc) in the reverse micelles. The second monomer could, for example, be any second monomer as described herein. Such a reaction of a second monomer with a first monomer allows for the polyanionic particle size and morphology to be determined by the corresponding properties of the precursor reverse micelle. In one embodiment, the particle may have an aqueous core, or more specifically a water core, that may be accessible after copolymerization (e.g., crosslinking) or, alternatively, preloaded before copolymerization (e.g., crosslinking). The resulting copolymer may be a polyanion or a polycation, or may be neutral. The copolymer may, in one embodiment, be further derivatized. For example, metal ions such as Ag⁺, which are aimed towards particle characterization by electron microscopy, could be added, as well as Ag-organic composites. Similarly, any metal ion or metal-organic composite could be added to the particle during further derivatization. In one embodiment, the hyperbranched polymeric particle (e.g., CDA gluc-NP) (cetyldimethylammonium acetamide gluc-NP) may react with preformed silver nanoparticles to form superstructures.

The outward orientation of carboxylate groups, in one embodiment, may be sustained after copolymerization (e.g., hydroxyl crosslinking) in organic media. In an aqueous medium, the carboxylate and carboxylic acid groups could be exposed to water accompanied by some structural rearrangement that entail micelle formation by the charged entity (e.g., surfactant) and cluster formation by the polymeric particle (e.g., gluc-NP). In one embodiment, the first monomer (e.g., gluc) paired with the charged entity (e.g., CDA) is freely soluble in chloroform.

One or more catalysts may optionally be used in the copolymerization reaction. For instance, a disulfide forming catalyst, FeNTA, can be used for disulfide formation (Walters et al., “The Formation of Disulfides by the [Fe(nta)Cl₂]²⁻ Catalyzed Air Oxidation of Thiols and Dithiols,” Inorg. Chim. Acta 359:3996 (2006), which is hereby incorporated by reference in its entirety).

In one embodiment, all the terminal groups of all compounds or all molecular complexes the copolymerization reactions between the first and second monomers have been linked with neighboring compounds or molecular complexes. Copolymerizing compounds or molecular complexes therefore form a completely enclosed hyperbranched polymer shell shown by the hyperbranched polymeric units. In such an embodiment, there are no terminal reactive groups on the polymer shell and, thus, the compounds do not react with neighboring complexes.

In another embodiment, not all terminal groups of all compounds or all molecular complexes have been copolymerized with the neighboring compounds or molecular complexes. In this example, the hyperbranched polymer nanoparticle then contains not only the hyperbranched polymeric units, but also the terminal compounds or terminal molecular complexes which have terminal functional groups that have not been linked with the neighboring compounds or molecular complexes. These terminal compounds or terminal molecular complexes then can have one, two or three groups linked with the hyperbranched polymeric units. Such reactive groups may react with neighboring complexes.

The anionic monomer glucuronate (gluc) with four reactive hydroxyl groups may be suited to form branched polymers and the resulting particle, gluc-NP, may be assembled as a two-dimensional hyperbranched polymeric shell. By virtue of the gluc carboxylate group, the poly-gluc particle has the capacity to bind metal ions and metal nanoparticles. The crosslinked product CDA gluc-NP can be dispersed in either organic media or water.

Polymerization using a hydroxyl coupling agent such as epichlorohydrin precludes the use of alcohol cosurfactants that are normally employed to stabilize cetyltrimethylammonium reverse micelles. Therefore, cationic surfactants such as C₁₂-C₁₈ alkyl dimethylammonium acetamide, C₁₂-C₁₈ alkyl trimethylammonium, and mixtures form stable reverse micelles that may be used with glucuronate in the absence of cosurfactant. Walters et al., “Amide-Ligand Hydrogen Bonding in Reverse Micelles,” Inorg. Chem. 44:1172-4 (2005); Mehltretter C. L., “Preparation of Esters, Hydrazides, and Amides of Carboxymethyldimethyl Long-Chain Aliphatic Ammonium Chlorides,” Journal of the American Oil Chemists' Society 44:219-20 (1967); and Shelton et al., “Quaternary Ammonium Salts as Germicides. II. Acetoxy and Carbethoxy Derivatives of Aliphatic Quaternary Ammonium Salts,” J. Am. Chem. Soc. 68:755-7 (1946), all of which are hereby incorporated by reference in their entirety.

The term “alkyl” refers to an aliphatic hydrocarbon group which may be linear, branched, or cyclic hydrocarbon structures and combinations thereof. Representative alkyl groups are those having 20 or fewer carbon atoms, for instance, methyl, ethyl, n-propyl, i-propyl, n-butyl, s-butyl, t-butyl, n-pentyl, i-pentyl, n-hexyl, and the like. Lower alkyl refers to alkyl groups having about 1 to about 6 carbon atoms in the chain. Branched alkyl means that one or more lower alkyl groups such as methyl, ethyl or propyl are attached to a linear alkyl chain.

As part of the characterization of gluc-NP, its reaction with Ag⁺ ions and Ag nanoparticles may be exploited. Silver ions serve this project in two distinct capacities; first Ag⁺ provides a stain for the TEM characterization of gluc-NP. Second the extent of Ag⁺ uptake serves as a measure of the capacity of gluc-NP to bind metal ions. It was found that CDA gluc-NP reacts with preformed silver nanoparticles to assemble nanoparticle superstructures.

In one embodiment, the charged entity is derived from a surfactant. The charged entity, in one embodiment, may be derived from a cationic surfactant. Any surfactant known to one skilled in the art for forming nanoparticles can be used to prepare the surfactant associated to the surface of the encapsulated core region. Suitable cationic surfactants include, but are not limited to, C₁₂-C₁₈ alkyl dimethylammonium acetamide, C₁₂-C₁₈ alkyl trimethylammonium, and mixtures thereof. Examples of such cationic surfactants are cetyldimethylammonium acetamide, octadecyl-dimethylammonium acetamide, tetradecyl-dimethylammonium acetamide, dodecyl-dimethylammonium acetamide, cetyltrimethylammonium, octadcecyl-trimethylammonium, tetradecyl-trimethylammonium, dodecyl-trimethylammonium, dimethyldioctadecylammonium, dioctadecyldimethylammonium, and mixtures thereof. Suitable sources of these cations of the cationic surfactant include, but are not limited to, alkyltrimethylammonium salts: such as cetyl trimethylammonium bromide (CTAB) or cetyl trimethylammonium chloride (CTAC); cetylpyridinium chloride (CPC); dimethyldioctadecylammonium chloride; dioctadecyldimethylammonium bromide (DODAB); cetyldimethylammonium acetamide bromide; or other cationic surfactant alike, including lipids. Alternatively, the surfactant may be benzyl hexadecyl dimethyl ammonium chloride (BHDC). The nanoparticle may further comprise a charged entity that is derived from at least one neutral surfactant. The molecular chains of this neutral surfactant can be interspersed with the individual molecules of cationic surfactant. For instance, the neutral surfactant can be a polyethelene glycol lauryl ether. In one embodiment, the neutral surfactant is Brij L23, which is a PEG-containing diblock copolymer surfactant. The charged entity may be paired with the first monomer to form a reverse micelle. That charged pair may then, in one embodiment, be reacted with a second monomer such as epichlorohydrin to form a copolymerized nanoparticle as illustrated in FIG. 5.

In another embodiment, the charged entity may be a charged substituent. Examples of charged substituent include, but are not limited to, a metal selected from the group consisting of silver, gold, copper, platinum, iron, manganese, cobalt, and mixtures thereof. In one example of such an embodiment, the charged substituent is reacted with the first monomer (e.g., glucoronate groups) of CDA gluc-NP by the addition of an aqueous medium. The aqueous medium may be, for example, AgNO₃. In such a system, the metal redox reaction may convert the first monomer (e.g., glucuronic acid) to a monoprotic form of glucaric acid. The monoprotic glucaric acid may bind up to two equivalents of the elemental metal that is produced in the reaction. Glucaric acid is produced by the reaction of glucuronic acid with silver ions. The general reaction of silver ions with certain sugars is used in the “Tollens test” for sugar, which is known to those skilled in the art.

The ratio of the first monomer to the charged entity can vary. For example, in one embodiment, the ratio of the first monomer to the charged entity is in the range of 3:1 to 1:3. For example, the ratio of the first monomer to the charged entity may be 3:1, 3:2, 3:3, 2:1, 2:2, 2:3, 1:1, 1:2, and 1:3. Additional examples of ratios of the first monomer to the charged entity may be 1:4, 1:5, or 1:6.

The nanoparticles and the size of their core region can be characterized by various methods, including but not limited to, small angle x-ray scattering (SAXS), neutrons scattering, transmission electron microscopy (TEM), and dynamic light scattering (DLS).

In one embodiment, the nanoparticle further includes a core material selected from the group consisting of water, dye molecules, drugs, inorganic ions, organic ions, other water soluble species, metals, and combinations thereof.

Dye molecules that may make up the core material may be include, but are not limited to, methylene blue, prussian blue, acridine orange, gentian violet, brilliant green, acridine yellow, quinacrine, trypan blue, and trypan red.

Exemplary drugs that may make up the core material include, but are not limited to, analgesic, antibacterial, anti-infective, anti-inflammatory, antiviral, antibiotic, anticholinergic, antidiabetic, antihistamine, antimicrobial, antifungal, antioxidant, chemotherapy, diuretic, enzyme replacement, and immunosuppressive drugs.

Inorganic ions that may comprise the core material include, for example, aluminum, barium, beryllium, calcium, chromium, copper, hydrogen, iron, lead, lithium, magnesium, manganese, potassium, mercury, silver, sodium, strontium, tin, zinc, bromide, chloride, fluoride, hydride, iodide, nitride, oxide, sulfide, carbonate, chlorate, chromate, dichromate, dihydrogen phosphate, hydrogen carbonate, hydrogen sulfate, hydrogen sulfite, hydroxide, hypochloride, monohydrogen phosphate, nitrate, nitrite, permanganate, peroxide, phosphate, sulfate, sulfite, superoxide, thiosulfate, metasilicate, and aluminum silicate.

Likewise, organic ions may be included in the encapsulated core. Examples of organic ions that may be used in accordance with the present invention include acetate, formate, oxalate, sugar acid carboxylate form, and cyanide.

The core material may also contain a water soluble species in the form of an alcohol. Alcohols that are linear or branched mono-alcohols from C2 to C6 may be useful as a core material. Examples of alcohols are methanol, ethanol, 1-butanol, 2-butanol, 3-methyl-1-butanol, 2-methyl-1-propanol, 1-pentanol, 1-propanol, 2-propanol, propanol, butanol, pentanol, hexanol, and heptanol, and an ammonium, nitrate, acetate, chloride, and sulfate salt thereof, and any mixture thereof.

In one embodiment, the nanoparticle further includes one or more metals in the core region. The metal in the nanoparticle may be selected from the group consisting of gold, silver, copper, platinum, iron, manganese, cobalt, and mixtures thereof. The iron may be present in the nanoparticle as an iron oxide selected from the group consisting of FeO, Fe₂O₃, Fe₃O₄, and mixtures thereof.

In another embodiment, the nanoparticle further includes one or more ions in the core region. The ions may be selected from ions of the lanthanide series, ions of the transition metal series, and mixtures thereof. Suitable lanthanides or transition metal ions include ions of iron, gadolinium, europium, manganese, dysprosium, ytterbium, lanthanum, lutetium, and mixtures thereof.

The encapsulated core of the nanoparticle may have varying sizes. For example, the size of the core may be around 4 nm, less than 4 nm, or more than 4 nm. In one embodiment, the core may be between 3 to 5 nm. The nanoparticle itself may also have varying sizes, ranging from 5 nm to 500 nm, preferably between 5 and 150 nm. In one embodiment, the nanoparticle may be at least 4, at least 5, at least 6, at least 7, at least 8, at least 9, at least 10, at least 11, at least 12, at least 13, at least 14, at least 15, at least 16, at least 17, at least 18, at least 19, or at least 20 mm in diameter. The method is facile having been implemented with polysaccharides, which yield non-uniform particle sizes. Reverse micelles tend toward size uniformity as occurs in this invention, which yields uniform particles of 14 nm in diameter (w=11.9). Moreover, the particles may be transferred from organic media to aqueous media while retaining solubility.

The compounds of the nanoparticle can be analyzed by appropriate means. For example, gas chromatographic analysis and high-performance liquid chromatography (HPLC), in particular with a light scattering detector, on a silica column, in the presence of an eluent, e.g. isocratic acetonitrile, may be used. Gas chromatography can also be used.

Copolymerization between functional groups of monomers leads to stable reverse copolymer formation without inter-micelle crosslinking to form undesirable particle dimers, trimers, or clusters. The process may be described as emulsion surface polymerization (ESP). Through ESP, a large variety of anionic monomer compounds can be employed for formation of nanoparticles or surfaces.

In one embodiment, the nanoparticle may also include a plurality of polyethylene glycol (PEG) molecules attached to the nanoparticle. The PEG groups form a corona around the metal core. The PEG corona prevents particle aggregation prior to administration to a subject and renders the particle largely undetectable by the reticuloendothelial system after administration. Such particles are long-circulating in the blood stream, which can reduce the amount to be injected, extend the duration of MRI data acquisition, and provide more detailed images.

The product may also include a plurality of alkane thiol or disulfide containing molecules attached to the nanoparticle. The attachment of alkane thiols is useful for adjusting the concentration of lanthanide complexes on the particle surface. The addition of alkane thiols also permits control of the solubility of the particle. Simple alkane thiols lower the solubility of the particles (constructs). Complex alkane thiols such as polyethylene glycol thiols increase the water solubility of the constructs and enhance their evasion of the immune (reticuloendothelial) system, which allows the particles to persist in the circulatory system thereby enhancing tissue targeting and image construction.

The product may also include a plurality of peptides containing cysteine attached to the particle. Peptides serve to alter the targeting properties and solubility of the construct. The product may also include a plurality of radioisotopes attached to the particle. Radioisotopes allow, for example, monitoring of delivery of the particles as well as delivery of drugs or other molecules through the targeted constructs such as peptides.

In one embodiment, the present invention is a water-in-oil reverse micelle that is assembled using a cationic hydrogen bonding surfactant, alkyl dimethylammonium acetamide (ADA) and cetyltrimethylammonium (CTA). The reverse micelle may contain a deprotonated sugar acid (SA) that serves as an anionic counterion and monomer that is crosslinked to form a polyanionic nanoparticle. The crosslinker may be epichlorohydrin to form ether linkages, or a diacid chloride to form an organic ester link, or sodium trimetaphosphate to form inorganic ester linkages between the sugar acid monomer units. The crosslinking step furnishes a porous nanocapsule that contains water.

Another aspect of the present invention relates to a nanoparticle. The nanoparticle includes a shell formed from a first monomer and a second monomer different from the first monomer. The first and second monomers are copolymerized, and the shell encapsulates a core region and has a neutral charge.

This aspect of the invention is carried out in accordance with the aspects described above. For example, in one embodiment the first monomer and the second monomer are covalently linked. In other embodiments, the core region may contain a core material selected from the group consisting of water, dye molecules, drugs, inorganic ions, organic ions, other water soluble species, and metals, as described above.

Another aspect of the present invention relates to a dispersion. The dispersion includes a nanoparticle in accordance with the present invention and a medium selected from the group consisting of water, methanol, dimethyl sulfoxide, chloroform, methylene chloride, and mixtures thereof. The nanoparticle is dispersed in the medium.

The dispersion medium may be produced, for example, by use of an easily dispersible colloid. In one embodiment, the dispersion may contain a sufficient concentration of nanoparticles to allow administration of an effective amount of the nanoparticles to a subject in need thereof; and yet not too great a concentration of nanoparticles such that the dispersion is too viscous or unstable. For example, the dispersion may comprise a range of about 0.1 to about 40 weight %. For example, the dispersion may be within the range of about 0.5 to about 20 weight % or within the range of about 1 to about 10 weight % of the nanoparticles, based on the weight of the dispersion.

The dispersion could further contain at least one stabilizer. The stabilizer may be adsorbed on the surfaces of the nanoparticles. The nanoparticles may be dispersed into a liquid medium, and the stabilizer may be employed as an adjuvant to aid in the wetting and/or the separation of the individual nanoparticles during the dispersion process. The ability of a stabilizer to aid in the wetting and/or the separation of the individual nanoparticles may be determined by comparing the dispersion processes for a composition containing the stabilizer and a control composition without the stabilizer. The ability of a stabilizer to aid in the wetting and/or separation of individual nanoparticles may be indicated by shorter dispersion times to obtain dispersions of the same average particle diameter, or smaller average particles diameters for the same dispersion time, under similar processing conditions. Alternatively, the stabilizer may be employed to promote stability of the dispersed nanoparticles in the liquid medium, preferably an aqueous medium. The ability of a stabilizer to promote the stability of the nanoparticles may be determined by less settling of the nanoparticles after a period of 24 hours at 20° C. for the dispersion comprising the stabilizer compared to a control dispersion without the stabilizer. Further, the stability may also be ascertained by the absence or near absence of agglomerates or particles greater than 200 nm.

Another aspect of the present invention relates to a method of making a nanoparticle. The method includes providing a first monomer having a first charge and providing a charged entity having a second charge of opposite sign to the first charge. The first monomer is contacted with the charged entity in an aqueous medium under conditions effective to form a complex where the first monomer is associated with the charged entity. A second monomer is provided and contacted with the complex under conditions effective to form a shell of the first and second monomers which have been copolymerized. The shell encapsulates a core region containing an aqueous medium.

The first monomer, second monomer, and charged entity are in accordance with the aspects described above.

In one embodiment, the charged entity is a surfactant, or in particular, a cationic surfactant selected from the group consisting of C₁₂-C₁₈ alkyl dimethylammonium acetamide, C₁₂-C₁₈ alkyl trimethylammonium, and mixtures thereof. Examples of such cationic surfactants are cetyldimethylammonium acetamide, octadecyl-dimethylammonium acetamide, tetradecyl-dimethylammonium acetamide, dodecyl-dimethylammonium acetamide, cetyltrimethylammonium, octadcecyl-trimethylammonium, tetradecyl-trimethylammonium, and dodecyl-trimethylammonium. For example, the charged entity may be cetyldimethylammonium acetamide (“CDA”).

In one embodiment, the first monomer may be a deprotonated sugar acid comprising three or more hydroxyl groups as described herein. Linking of the hydroxyl groups is enabled by the use of a charged entity (e.g., a cationic surfactant) that assembles the monomers in close proximity in solution without cosurfactants. In another embodiment, the first monomer is selected from the group consisting of carboxylic acids, amines, alcohols, thiols, aldehydes, ketones, ethers, esters, nitriles, imides, or any salt thereof. In another embodiment, the first monomer is an anionic monomer and may be a sugar acid selected from the group consisting of aldonic acids, ulosonic acids, uronic acids, and aldaric acids. The sugar acid may be, for example, the uronic acid glucuronic acid. The first monomer may be glucuronic acid (gluc-H) in its carboxylate form (gluc) which can be paired with a charged entity such as a cationic surfactant like, but not limited to, CDA. The pair can assemble uniform reverse micelles in a solution. This scheme is merely an example of the present invention which is not limited to the use of gluc and CDA exclusively. Rather, gluc could be replaced with any first monomer as described above. Similarly, gluc could be paired with any charged entity as described above. CDA and glucoronate (i.e., glucuronic acid in its deprotonated form) (“gluc”) are shown in Scheme 1:

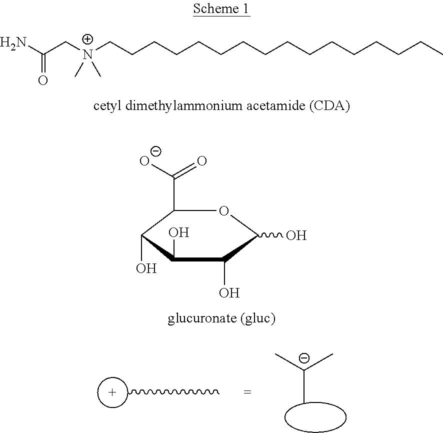

See Walters et al., “Amide-Ligand Hydrogen Bonding in Reverse Micelles,” Inorg. Chem. 44:1172-74 (2005); Mehltretter C. L., “Preparation of Esters, Hydrazides, and Amides of Carboxymethyldimethyl Long-Chain Aliphatic Ammonium Chlorides,” Journal of the American Oil Chemists' Society 44:219-20 (1967), both of which are hereby incorporated by reference in their entirety. In this embodiment, CDA⁺ is employed to form the salt CDA-glue. The CDA-gluc salt dissolves in organic solvents to form a microemulsion composed of reverse micelles. The volume occupied by anions may be dry (see Walters et al., “Amide-Ligand Hydrogen Bonding in Reverse Micelles,” Inorg. Chem. 44:1172 (2005), which is hereby incorporated by reference in its entirety) or may incorporate water in the manner of classical reverse micelles. The microemulsion serves as a precursor for the preparation of hyperbranched polymeric nanoparticles.

In one embodiment, the CDA-gluc salt forms a small spherical reverse micelle in chloroform when the amount of water: surfactant ratio (w value) is appropriate. The reverse micelle consists of the charged entity (e.g., a cationic surfactant) and a first monomer (e.g., an anionic sugar) whose hydroxyl groups are available for crosslinking reactions within the reverse micelle to form a hyperbranched polymeric nanoparticle. See, e.g., Schemes 2 and 3. Linking of the hydroxyl groups is enabled by the use of a charged entity (e.g., a cationic surfactant) that stabilizes the reverse micelle without cosurfactants.

In particular, Scheme 2 shows crosslinking and of gluc by sodium trimetaphosphate.

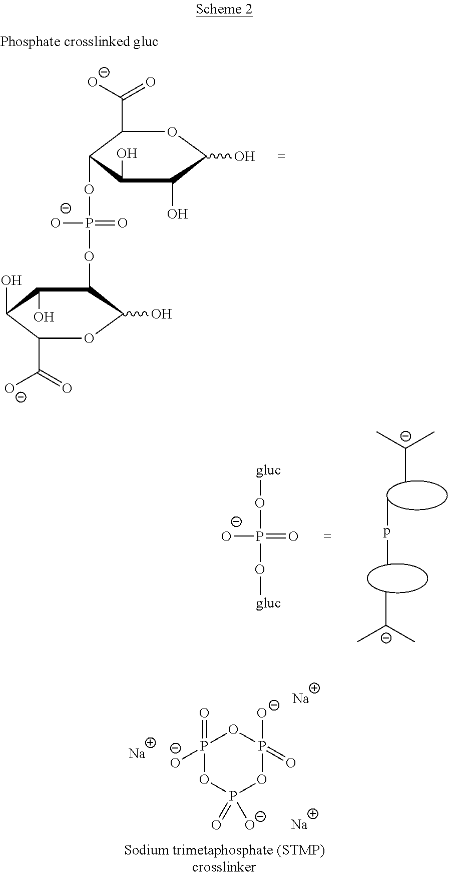

Scheme 3 provides a schematic example of one embodiment, where the a [Gd(gluc)3] complex is incorporated in a CDA-gluc reverse micelle followed by crosslinking.

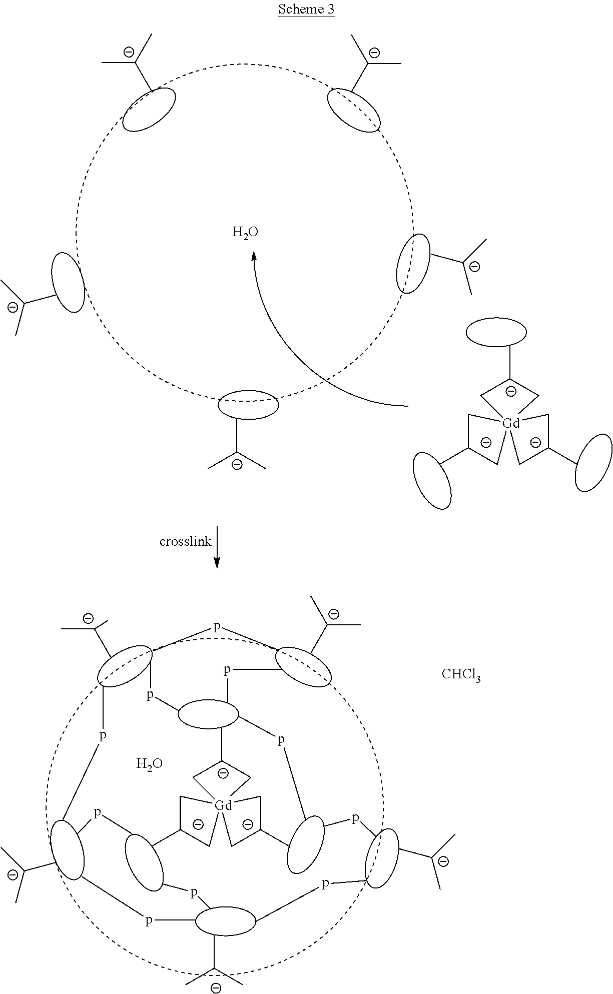

In one embodiment, the method further includes replacing the charged entity with a charged metal substituent. The charged metal substituent may be, but is not limited to, silver, gold, copper, platinum, iron, manganese, cobalt, and mixtures thereof. In another embodiment, the method further includes replacing the aqueous medium of the core region with a material. The material may be selected from the group consisting of water, dye molecules, drugs, inorganic ions, organic ions, other water soluble species, and metals, in accordance with those described above.

This synthetic approach can be applied to a vast array of monomers and can be of great commercial importance in the assembly of diagnostic, theranostic, or catalytic nanoparticles. Synthesis of the nanoparticles of the present invention is cost effective and could have limitless applications.

Another aspect of the present invention relates to a method of imaging. The method includes providing the nanoparticle of the present invention where the nanoparticle encapsulates an imaging agent in the core region. A subject to be imaged is provided and the nanoparticle is administered to said subject. An imaging procedure is conducted on said subject to whom the nanoparticles have been administered.

The nanoparticle of this aspect is configured and prepared as discussed above.

In one embodiment, the imaging procedure is, for example, magnetic resonance imaging (MRI), computerized tomography (CT), nuclear magnetic resonance (NMR) analysis, fluorescence imaging, positron emission tomography (PET), surfaced enhanced Raman imaging, radiologic imaging, and may entail the targeted delivery of radioisotopes. In another embodiment, the subject has cancer and the imaging procedure is targeted delivery of radioisotopes to tumors in the subject. In another embodiment, the imaging is carried out in conjunction with a real-time MRI or CT guided procedure. In particular, the procedure may be a surgical procedure such as balloon angioplasty or catheterization. Magnetic resonance (MR) imaging, in particular, is a critical medical diagnostic tool in human health. The use of MR contrast enhancement agents in MR imaging protocols has proven to be a valuable addition to the technique by improving both the quality of images obtained in an MR imaging procedure and the efficiency with which such images can be gathered. MR imaging, for example, relies on the application of a strong magnetic field to a recipient's body to generate images of tissue and bone structure. The magnetic field aligns proton (hydrogen atoms) spins within the subject's body. These atoms are then excited into resonance by an applied RF field. The atoms release energy as they exit their excited state. Protons spin excited state lifetimes vary as a function of tissue type. Proton spin excited state lifetimes are correlated with the signal intensity from the proton spins. Since the lifetimes are a function of the proton environment, the lifetimes of the excited states and hence the signal intensity is a function of the type of tissue in which the protons reside. These characteristics allow for magnetic resonance imaging by non-invasive methods.

The protons of tissue lesions are the same as those of the surrounding healthy tissue. As a result, the lesions cannot be detected without the addition of an MRI contrast agent that specifically accumulates in the lesion. The contrast agent has magnetic properties (paramagnetic) that decreases the excited state lifetimes of protons in the vicinity of the agent, which gives rise to a signal that is distinct (contrasts) from that of the surrounding tissue. This release of energy is detected by a receiver and utilized to create an MRI image. The present aspect allows the generation of one or more MRI images from a subject by scanning a one or more scan slices of the recipient with an MRI machine.

The particles that are formed may be designed to serve as dual imaging agents for combined MRI and photoacoustic tomography (PAT). The tomographic applications can be realized with the incorporation of Prussian blue, a metal complex phase or methylene blue, an organic dye. The MRI function may be installed with the incorporation of gadolinium (Gd) or manganese (Mn) complexes. In one formulation, these two classes of imaging agents can be combined within a single particle capsule for dual imaging applications (MRI-PAT). In a second formulation, the agents may be placed separately in particle capsules allowing the flexible option of combining particles with different imaging capacities as a cocktail. Stock solutions containing three or four particles with distinct and different imaging modalities for diagnostic purposes could be used. Each of the agents could be enclosed in a particle with identical capsule exteriors which should render them similar in their physiological properties.

Lanthanoid ions can be selected for diamagnetism (La³⁺) for NMR analyses, fluorescence properties (Eu³⁺) or MRI contrast (Gd³⁺). Main group isotopes ⁶⁸Ga³⁺ or ¹¹¹In³⁺ can be incorporated in the monomers for PET.

Another aspect of the present invention relates to a method of delivering drugs. The method includes providing a nanoparticle of the present invention with a drug encapsulated in the core and a subject to be treated. The nanoparticle is administered to the subject under conditions effective to deliver drugs.

The nanoparticle of this aspect of the invention is carried out in accordance with the previously described aspects.

The size of the hyperbranched shell encapsulating solid lipid nanoparticle for drug delivery typically ranges from 50-150 nm, or from 50-100 nm.

Any therapeutic drug known by those of skill in the art to have therapeutic activity can be contained in the solid lipid nanoparticles. Suitable therapeutic agents include, but not limited to, chemicals, proteins, peptides, nucleic acids (e.g., DNA, RNA, siRNA), nucleic acid analogues, nucleotides, oligonucleotides or sequences, peptide nucleic acids(PNA), aptamers, antibodies or fragments or portions thereof (e.g., paratopes or complementarity-determining regions), antigens or epitopes, hormones, hormone antagonists, cell attachment mediators (such as RGD), growth factors or recombinant growth factors and fragments and variants thereof, cytokines, enzymes, antioxidants, antibiotics or antimicrobial compounds, anti-inflammation agents, antifungals, viruses, antivirals, toxins, prodrugs, drugs, dyes, amino acids, vitamins, chemotherapeutic agents, and small molecules. The agent may also be a combination of any of the above-mentioned therapeutic agents.

In one embodiment, the therapeutic agent is an antibiotic or anti-tumor agent. Exemplary antibiotic agents include, but are not limited to, doxorubicin; actinomycin; aminoglycosides (e.g., neomycin, gentamicin, tobramycin); β-lactamase inhibitors (e.g., clavulanic acid, sulbactam); glycopeptides (e.g., vancomycin, teicoplanin, polymixin); ansamycins; bacitracin; carbacephem; carbapenems; cephalosporins (e.g., cefazolin, cefaclor, cefditoren, ceftobiprole, cefuroxime, cefotaxime, cefipeme, cefadroxil, cefoxitin, cefprozil, cefdinir); gramicidin; isoniazid; linezolid; macrolides (e.g., erythromycin, clarithromycin, azithromycin); mupirocin; penicillins (e.g., amoxicillin, ampicillin, cloxacillin, dicloxacillin, flucloxaciUin, oxacillin, piperacillin); oxolinic acid; polypeptides (e.g., bacitracin, polymyxin B); quinolones (e.g., ciprofloxacin, nalidixic acid, enoxacin, gatifloxacin, levaquin, ofloxacin, etc.); sulfonamides (e.g., sulfasalazine, trimethoprim, trimethoprim-sulfamethoxazole (co-trimoxazole), sulfadiazine); tetracyclines (e.g., doxycyline, minocycline, tetracycline, etc.); monobactams such as aztreonam; chloramphenicol; lincomycin; clindamycin; ethambutol; mupirocin; metronidazole; pefloxacin; pyrazinamide; thiamphenicol; rifampicin; thiamphenicl; dapsone; clofazimine; quinupristin; metronidazole; linezolid; isoniazid; piracil; novobiocin; trimethoprim; fosfomycin; fusidic acid; or other topical antibiotics. Optionally, the antibiotic agents may also be antimicrobial peptides such as defensins, magainin and nisin; or lytic bacteriophage. The antibiotic agents can also be the combinations of any of the agents listed above.

In one embodiment, the therapeutic agent is doxorubicin. Doxorubicin is an anthracycline antibiotic and anti-tumor agent that intercalates DNA. It is effective against cancers that cause solid tumor formation as well as those that cause hematological malignancies (Booser & Hortobagyi, “Anthracycline Antibiotics in Cancer Therapy. Focus on Drug Resistance,” Drugs 47:223 (1994); Serpe et al., “Cytotoxicity of Anticancer Drugs Incorporated in Solid Lipid Nanoparticles on HT-29 Colorectal Cancer Cell Line,” Eur. J. Pharm. Biopharm. 58:673 (2004), which are hereby incorporated by reference in their entirety). Its administration in solution as doxorubicin.HCl (DOX.HCl) causes many side effects, the most serious of which are cardiotoxicity, and myelosuppression (Subedi et al., “Preparation and Characterization of Solid Lipid Nanoparticles Loaded with Doxorubicin,” Eur. J. Pharm. Sci. 37:508 (2009); Zara et al., “Pharmacokinetics of Doxorubicin Incorporated in Solid Lipid Nanospheres (SLN),” Pharmacol. Res. 40:281 (1999), all of which are hereby incorporated by reference in their entirety). Improved safety can be achieved when doxorubicin is administered in lanthanoid-DOTA derivatives.

The nanoparticle described herein can be administered by various routes known to skilled in the art.

The nanoparticles allow absorption of the compounds to be delivered across mucosa, preferably across mouth, nasal and/or rectal mucosa. Also, nanoparticles of the present invention provide an important bioavailability with low variability of absorption. Another possible route of administration is through intravenous administration. With normal kidney function, the gadolinium complexes should be eliminated from the circulatory system within 2-3 hours to avoid the accumulation of free Gd³⁺ ions, which can cause nephrogenic systemic fibrosis (NSF) (Bongartz, G., “EDITORIAL REVIEW Imaging in the Time of NFD/NSF: Do We Have to Change,” Magn. Reson. Mater. Phy. 20:57 (2007); Penfield & Reilly, “What Nephrologists Need to Know about Gadolinium,” Nat. Clin. Pract. Nephr. 3:654 (2007), all of which are hereby incorporated by reference in their entirety).

Another aspect of the present invention relates to a method of delivering a high concentration of contrast enhancing and/or imaging agents. The method includes providing a nanoparticle in accordance with the present invention with a contrast enhancing and/or imaging agent encapsulated in the core and providing a subject to be treated. The nanoparticle is then administered to the subject.

The nanoparticle of this aspect of the invention is configured and prepared as discussed above.

The contrast enhancement agents of the present invention may be employed in tumor and blood clot imaging applications, in vivo assays, wound healing assessment, angiogenesis, and imaging tumor boundary regions. The contrast enhancement agent used according to the methods of the present invention are suitable for use as imaging agents for magnetic resonance (MR) screening of human subjects for various pathological conditions. As will be appreciated by those of ordinary skill in the art, MR imaging has become a technique of critical importance to human health.

Contrast enhancement agents used according to the method of the present invention may include a paramagnetic iron center that may be readily excreted by human subjects and by animals and as such may be rapidly and completely cleared from the subject following the magnetic resonance imaging procedure. In addition, the contrast enhancement agents used according to the method of the present invention may enable the administration of lower levels of contrast enhancement agents to the subject relative to known contrast enhancement agents without sacrificing image quality. Thus, in one embodiment, useful MR contrast enhancement using contrast agents according to the method of the present invention, is achieved at lower dosage level in comparison with known MR contrast agents. In an alternate embodiment, the contrast enhancement agents used according to the method of the present invention may be administered to a subject at a higher dosage level in comparison with known MR contrast agents in order to achieve a particular result. Higher dosages of the contrast enhancement agents of the present invention may be acceptable in part because of the enhanced safety of iron-based contrast enhancement agents, and improved clearance of the contrast enhancement agent from the subject following an imaging procedure. In one embodiment, the contrast enhancement agent is administered in a dosage amount corresponding to from about 0.001 to about 5 millimoles per kilogram weight of the subject. As will be appreciated by those of ordinary skill in the art, contrast enhancement agents used according to the method of the present invention may be selected and/or further modified to optimize the residence time of the contrast enhancement agents in the subject, depending on the length of the imaging time required.

Contrast enhancing agents useful in the present invention include substances that affect the attenuation, or the loss of intensity or power, of radiation as it passes through and interacts with a medium. It will be appreciated that contrast enhancing agents may increase or decrease the attenuation. Contrast enhancing agents may be classified in various ways. In one classification, for example, iodinated contrast enhancing agents can be water soluble (e.g., monoiodinated pyridine derivatives, di-iodinated pyridine derivatives, tri-iodinated benzene ring compounds, and the like), water-insoluble (e.g., propyliodone and the like), or oily (e.g., iodine in poppy seed oil, ethyl esters of iodinated fatty acids of poppy seed oil containing iodine, and the like). In one embodiment, the contrast enhancing agents can contain iodine and may be called “iodinated.”

In one example, a grouping of iodinated contrast enhancing agents are water soluble. Water soluble iodinated contrast enhancing agents can be derivatives of tri-iodinated benzoic acid. These compounds can have one or more benzene rings and may be ionic or nonionic. Suitable, nonionic compounds include, but are not limited to, metrizamide, iohexol, iopamidol, iopentol, iopromide, ioversol, iotrolan, iodoxanol and others. Further examples of contrast enhancement agents include any agent in experimental and clinical imaging research such as the water soluble chelate gadolinium-DTPA. When a contrast enhancement agent of the present invention is administered to a subject, such as a human, the prescribing physician will ultimately determine the appropriate dosage for a given human subject, and this can be expected to vary according to the weight, age and response of the individual as well as the nature and severity of the subject's condition. In one example, the contrast enhancing agents described herein are contrast enhancing agents for X-ray imaging or used for X-ray CT. In one embodiment, the contrast enhancing agents used herein are nonradioactive.

The present invention may be further illustrated by reference to the following examples.

### EXAMPLES

The following examples are intended to illustrate, but by no means are intended to limit, the scope of the present invention as set forth in the appended claims.

### Example 1

### Materials and Methods

Silver nitrate, D-glucuronic acid and anhydrous methanol were obtained from Sigma Aldrich. Epichlorohydrin and N, N-dimethylhexadecylamine were obtained from TCI America. Triethylamine and 2-chloroacetamide were obtained Alfa Aesar. Potassium hydroxide was obtained from VWR international. Chloroform, acetone and diethyl ether were obtained from Pharmco-AAPER. All deuterated solvents were obtained from Cambridge Isotope Laboratories, Inc. Reagents and solvents were used without further purification.

Anhydrous Proton NMR data were obtained on a Bruker Avance-400 MHz NMR spectrometer. Thermogravimetric analyses (TGA) were carried out on a Texas Instruments SDT Q600 and a Perkin-Elmer Pyris 1. TGA profiles were recorded by using a Universal Analysis program. Electrospray ionization mass spectrometric data were obtained using an Agilent 1100 Series Capillary LCMSD Trap XCT MS spectrometer. Infrared data were obtained on a Nicolet 750 spectrometer. UV/Vis spectrum was obtained on a Lambda 950 spectrometer and processed on included Perkin-Elmer software. For TEM, samples were deposited on carbon coated copper grids examined under a Philips CM-12 electron microscope. The micrographs were recorded on a Gatan 1 k 1 k digital camera.

Scanning electron microscopy was carried out using a Merlin (Carl Zeiss) field-emission SEM typically operating at 3 keV. Samples were prepared by placing a drop of dilute particles dispersed in chloroform on a copper 300 mesh carrier grids covered with carbon-coated Formvar. The solvent was allowed to evaporate in air at room temperature. The grid was then coated with a 2.5 nm layer of platinum. The grid was then mounted on an aluminum SEM stub using a conductive tape.

Atomic force microscopy (AFM) images were acquired using an Asylum MFP-3D-SA atomic force microscope in tapping mode using a Bruker SNL-10 D-triangular shaped silicon nitride cantilever for all measurements (spring constant=0.6 N/m, resonance frequency=18 kHz). The particles were prepared by depositing a chloroform dispersion of the particles on ultra-flat mica surface. The mica was mounted on a glass slide using a conductive tape. The images were collected at a scan rate of 1.0 Hz. AFM images were analyzed using the software system Gwyddion. Necas et al., “Gwyddion: An Open-Source Software for SPM Data Analysis,” Cent. Eur. J. Phys. 10:181-8 (2012), which is hereby incorporated by reference in its entirety.

For X-ray powder diffraction studies data were collected on a Bruker D8 DISCOVER GADDS microdiffractometer equipped with a VANTEC-2000 area detector in a Φ to rotation method. The X-ray generated from a sealed Cu tube is monochromated by a graphite crystal and collimated by a 0.5 mm MONOCAP (λ, Cu-Kα=1.54178 Å). The sample-detector distance is 150 mm, and the exposure time is 300 seconds per run. Data were integrated by the XRD2EVAL program in the Bruker PILOT software. 2009 PILOT: (Madison, Wis.: Bruker AXS Inc.) pp Program for Bruker D8 DISCOVER X-ray Diffractometer Control, which is hereby incorporated by reference in its entirety. The raw file was converted by the UXD format by the DIFFRAC^{plus} FileExchange (2009 DIFFRACplus FileExchange: (Madison, Wis.) p Software Package for Powder Diffraction, which is hereby incorporated by reference in its entirety) which was later analyzed by the WINPLOTR program (2009 WinPLOTR. p Windows tool for powder diffraction patterns analysis, which is hereby incorporated by reference in its entirety).

Samples for dynamic light scattering are prepared by dissolving 1 mg of sample into 4 mL of solvent. The solution was then filtered through a 0.45 μm syringe filter into quartz cuvette with a path length of 2 cm. The measurements are obtained on a Malvern Zetasizer Nano.

### Example 2

### Cetyldimethyl Ammonium Acetamido Chloride (CDAC1)

The procedure follows earlier work by Walters and coworkers. Walters et al., “Amide-Ligand Hydrogen Bonding in Reverse Micelles,” Inorg. Chem. 44:1172-4 (2005), which is hereby incorporated by reference in its entirety. 2-chloroacetamide (1 g, 10.7 mmol) and N,N-dimethylhexadecylamine (3.6 mL, 10.7 mmol) were stirred and refluxed for 18 hours in acetonitrile. On cooling to room temperature a white crystalline product precipitated. The precipitate was collected by vacuum filtration. The crude product was then washed with diethyl ether and dried under vacuum to obtain a white powder. Yield: 3.65 g, 94%.

### Example 3

### Acetamido Cetyl Dimethyl Ammonium Hydroxide (CDAOH)

A solution of potassium hydroxide (100 mg, 1.78 mmol) in 5 mL dry methanol was combined with CDAC1 (646 mg, 1.78 mmol) in 5 mL dry methanol and stirred at room temperature for 1 hour during which KCl salt was produced as a white precipitate. KCl was removed by benchtop centrifugation. The methanol supernatant was removed by rotary evaporation and the product was washed with diethyl ether, collected by filtration and dried under vacuum leaving a fine white powder. Yield: 614.1 mg, 82%.

### Example 4

### Acetamido Cetyl Dimethyl Ammonium Glucuronate (CDA-Gluc)

Glucuronic acid (100 mg, 0.515 mmol) and CDAOH (177 mg, 0.515 mmol) were combined in 25 mL of anhydrous methanol and stirred for 1.5 hours at room temperature. Methanol was removed by rotary evaporation and chloroform was added to dissolve the crude product. The solution was dried by stirring for a few minutes over anhydrous sodium sulfate following which the chloroform solution was decanted into a separate vessel, filtered and then evaporated to dryness. The initially gel-like product was triturated under ether to form a white powder that was isolated after pouring of the ether and drying the product under vacuum. Yield: 230.66 mg, 86%.

### Example 5

### Hyperbranched Acetamido Cetyl Dimethyl Ammonium Glucuronate (CDA Gluc-NP)

CDA gluc (100 mg, 0.192 mmol), CDAOH (132 mg, 0.384 mmol) and deionized water (41.0 μL, 2.28 mmol) were combined with stirring in 8 mL chloroform to form reverse micelles. After 10 minutes epichlorohydrin (30.1 μL, 0.384 mmol) was added and the solution was allowed to stir at 40° C. for 18 hours. Chloroform was then removed under vacuum and the product was washed twice with ether leaving a gel. To remove excess reagents and biproducts the crude gel was dissolved into 5 mL of deionized water and filtered by centrifugation (Pall Microsep Advance, 30K cutoff) for 15 minutes at 3750 RPM and 25° C. The product was lyophilized and collected. Yield: 108.71 mg, 89.44%

### Example 6

### Gluc-NP Silver Composite (Gluc-NP Ag)

CDA gluc-NP (5 mg, 7.90 μmol) and excess AgNO₃ (6 mg, 35.2 μmol) were dissolved into 10 mL of water in a covered vial. The solution was stirred at room temperature for 18 hours. The solution was then filtered (Microsep™ tube) by centrifugation for 15 minutes at 3750 RPM and 25° C. The membrane-retained product was collected using a pipette and the water was removed by lyophilization leaving a brown powder. Yield: 2.90 mg, 66.39%.

### Example 7

### Silver Myristate (Ag-Myr)

The procedure was modified from the method reported by Yamamoto et al. Yamamoto et al., “Size-Controlled Synthesis of Monodispersed Silver Nanoparticles Capped by Long-Chain Alkyl Carboxylates From Silver Carboxylate and Tertiary Amine,” Langmuir 22:8581-6 (2006), which is hereby incorporated by reference in its entirety. Silver nitrate, AgNO₃, (100 mg, 0.5886 mmol) was combined with tetradecanoic (myristic) acid (134 mg, 0.5886 mmol) in a test tube to which 2 mL triethylamine was added. The solution was heated to 80° C. and stirred at 500 RPM for 2 hours. The solution turned brown within 10 minutes. After 2 hours, the triethylamine was removed by rotary evaporation and the crude product was washed twice with acetone and collected by centrifugation after each wash. The pellet was dissolved in chloroform and the solution was vacuum filtered to remove insoluble byproducts. Chloroform was removed by evaporation. The resulting solid was washed with acetone and dried under vacuum, leaving a lustrous purple powder. Yield: 43.17 mg, 68%.

### Example 8

### Hyperbranched Acetamido Cetyl Dimethyl Ammonium Glucuronate Silver Composite (CDA Gluc-NP Ag)

A stock 4.47 mM Ag-Myr solution in chloroform was prepared by dissolving Ag-Myr (15.0 mg, 0.0447 mmol) in 10 mL of chloroform. A stock 6.34 mM CDA gluc solution in chloroform was prepared by dissolving (CDAgluc (33 mg, 0.0634 mmol) in 10 mL chloroform. The Ag-Myr solution (1.00 mL, 4.47 μmol) and the CDA gluc solution (0.638 μL, 4.47 μmol) were combined, diluted to 2 mL and stirred in a vial at room temperature overnight under ambient conditions. A dark precipitate was formed and was collected by centrifugation at 8000 RPM for 5 minutes. The pellet was then washed with chloroform and collected by centrifugation until the chloroform supernatant was clear. After the final wash, the pellet was collected and dried under vacuum leaving a dark brown powder that was stored in a vial at room temperature.

### Example 9

### X-Ray Powder Diffraction (XRD)

A finely ground preparation of CDA glucNP was loaded in a 0.8 mm Kapton capillary and mounted on a magnetic base. In preparation for reflectance XRD data acquisition gluc-NP Ag powder was deposited on a silicon wafer (001 cut, 1 cm×1 cm) that was mounted on a pin stub by adhesion. Another piece of silicon wafer was used to press the powder gently to create a flat surface. The mount with the sample was carefully secured to the sample holder in the instrument.

### Example 10

### Reverse Micelles are Thermodynamically Stable and Uniform

Reverse micelles produced from simple surfactants are thermodynamically stable and uniform nanoscale structure. These characteristics have been exploited over several decades to produce polymers and inorganic nanoparticles through reaction in the water core of reverse micelles. Pileni M. P., “Fabrication and Properties of Nanosized Material Made by Using Colloidal Assemblies as Templates,” Cryst. Res. Technol. 33:1155-86 (1998), which is hereby incorporated by reference in its entirety. The incorporation of ionic monomers as part of a charged surfactant-counterion pair for the preparation of nanoparticles from linear polymers has been previously reported for the formation of linear polymeric nanoparticles. Hammouda et al., “Synthesis of Nanosize Latexes by Reverse Micelle Polymerization,” Langmuir 11:3656-9 (1995), which is hereby incorporated by reference in its entirety. The synthesis described here results in a hyperbranched polymeric nanoparticle from the reaction of epichlorohydrin with glucuronate of the reverse micelle, an a₂+b₄ system (as shown in FIG. 5). Flory, P. J., “Fundamental Principles of Condensation Polymerization,” Chem. Rev. 39:137-97 (1946) and Kricheldorf et al., “Biodegradable Hyperbranched Aliphatic Polyesters Derived From Pentaerythritol,” Macromolecules 41:5651-7 (2008), both of which are hereby incorporated by reference in their entirety. The advantage of this approach is that the acquired polyanionic particle size and morphology are determined by the corresponding properties of the precursor reverse micelle. The particle has a water core that may be accessible after crosslinking or preloaded before crosslinking. By virtue of its carboxylate rich surface, the resultant polyanion can be further derivatized. As shown in FIG. 5, the addition of Ag⁺ metal ions aimed towards particle characterization by EM was explored, and the acquisition of Ag-organic composites.

Based on earlier work with it was anticipated that the combination of charge and hydrogen bonding would sustain a close association of carboxylate with an ammonium head group of the surfactant. Walters et al., “Amide-Ligand Hydrogen Bonding in Reverse Micelles,” Inorg. Chem. 44:1172-4 (2005), which is hereby incorporated by reference in its entirety. The outward orientation of carboxylate groups would be sustained after hydroxyl crosslinking in organic media. In aqueous solution the carboxylate and carboxylic acid groups would plausibly be exposed to water accompanied by some structural rearrangement that entail micelle formation by the surfactant and cluster formation by gluc-NP.

The readily accessible salt cetyltrimethylammonium glucuronate (CTA-gluc) proved to be poorly soluble in chloroform. However when glucuronate (gluc) was paired with cetyldimethylammonium acetamide (CDA) the salt, CDA-gluc, proved to be freely soluble in chloroform. The CDA-gluc reverse micelles were characterized by spectroscopy and light scattering before and after the polymerization step.

### Example 11

### CDA-Gluc

Cetyldimethylammonium amide chloride, CDA-Cl was converted to the hydroxide form CDA-OH, by the reaction of CDA-Cl with KOH in anhydrous methanol leaving solid KCl as a biproduct that was removed by filtration. Pinho et al., “Solubility of NaCl, NaBr, and KCl in Water, Methanol, Ethanol, and Their Mixed Solvents,” J. Chem. Eng. Data 50:29-32 (2005), which is hereby incorporated by reference in its entirety. The surfactant CDA-gluc is then formed by the reaction of CDA-OH with gluc-H in anhydrous methanol. Standard workup yielded a mildly hygroscopic white crystalline powder. The product was easily dispersed in water and non-aqueous media that included MeOH, DMSO, and CHCl₃. Spectroscopic analyses were carried out in DMSO and CHCl₃.

### Example 12

### Glucuronate Hyperbranched Polymeric Particles

Reverse micelles formed spontaneously upon the addition of CDA-gluc to CHCl₃ containing water at the level of w=11.9. Dynamic light scattering measurements of this solution showed particles with an average diameter of 14 nm. Solubility properties dictate that gluc anions reside in the aqueous polar region of the reverse micelle and remain associated with CDA through the pairing of carboxylate with the cationic amide head group of CDA.

Sugar carboxylate monomers in the reverse micelles were converted to a hyperbranched polyether copolymer nanoparticle by the reaction of epichlorohydrin with the hydroxyl groups of sugar ions. The stoichiometry for this reaction is gluc:epichlorohydrin:CDA-OH or 1:2:2. Polymerization was initiated by adding CDA-OH as a base to the reverse micelle solution containing epichlorohydrin. As shown in Scheme 4, the polymer repeat unit of CDA gluc-NP may have the formula C₃₂N₂O₁₀H₆₀ with a molecular mass of 632.83 g/mol.

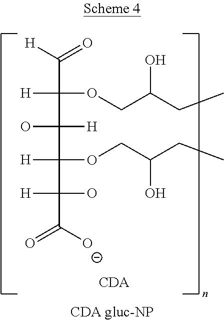

The carboxylate groups are not modified by polymerization and sustain charge pairing with the cationic head group of CDA. Charge pairing favors an assembly where the carboxylate groups of the sugars are oriented towards the amide groups of CDA. The surfactant CDA is known to form charge-pair hydrogen bonds in a previously reported dry reverse micelle system. Walters et al., “Amide-Ligand Hydrogen Bonding in Reverse Micelles,” Inorg. Chem. 44:1172-4 (2005), which is hereby incorporated by reference in its entirety. In hyperbranched nanoparticle CDA gluc-NP retains its average diameter of 14 nm as measured by DLS in chloroform.

The crosslinked nanoparticle is soluble in water, which suits it for the uptake of aqueous metal ions. Results from DLS measurements show an increase in particle size in water to 74 nm, which suggests that particle form clusters in water, perhaps in association with CDA micelles. The polyether product here is synthetically related to hyperbranched polyethers, particularly the hyperbranched polysaccharides. Satoh et al., “Synthesis of Hyperbranched Carbohydrate Polymers by Ring-Opening Multibranching Polymerization of Anhydro Sugar,” Macromol. Biosci. 7:999-1009 (2007) and Satoh, T. “Synthesis of Hyperbranched Polymer Using Slow Monomer Addition Method,” Int. J. Polym. Sci. (2012), both of which are hereby incorporated by reference in their entirety. However, unlike these earlier polymers the reverse micelle method yields a particle that is not dendritic in structure but is a reticulated capsule. Like dendrimers the reverse micelle based particle can be derivatized but its core capacity, and perhaps also its overall porosity, can be adjusted. Hence the nanoparticles described here add to the utility of hyperbranched systems.

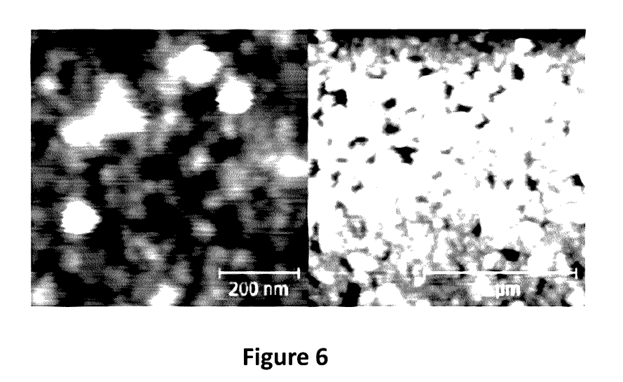

The CDA gluc-NP particles were deposited on mica and dried for AFM imaging (FIG. 6). Tapping mode images shows particles with a predominant height of 20 nm. A small number of particles sitting above a primary layer of particles on the mica surface resulted in the appearance of a few peaks at about 40 nm above the mica surface.

The CDA gluc and CDA gluc-NP assignments are based on 1-D and 2-D NMR. The integration of the 1-D NMR suggests that the degree of cross-linking is 87% by comparing the ratio of epichlorohydrin cross-linker protons to those of the CDA C₁₆ chain protons.

### Example 13

### Silver Ion Binding and Reduction in Water

Carboxylate groups in molecules and matrices routinely serve as binding sites for metal ions and metal containing nanoparticles. Yamamoto et al., “Size-Controlled Synthesis of Monodispersed Silver Nanoparticles Capped by Long-Chain Alkyl Carboxylates From Silver Carboxylate and Tertiary Amine,” Langmuir 22:8581-6 (2006); Cotton et al., ADVANCED INORGANIC CHEMISTRY (6th Edition: Wiley) (1998); and Goloverda et al., “Synthesis of Ultrasmall Magnetic Iron Oxide Nanoparticles and Study of Their Colloid and Surface Chemistry,” J Magn. Magn. Mater. 321:1372-6 (2009), all of which are hereby incorporated by reference in their entirety. Glucuronic acid (gluc-H) with its carboxylic acid group, four hydroxyl groups form a nanoparticle with a dense array of coordinating sites on its surface. Here, the carboxylates were employed to take up Ag⁺ ions as a stain for TEM characterization. In the process, a UV-vis active tag was acquired for the particles through a well-known redox reaction of Ag(I) with polysaccharides.

The literature is replete with green chemistry methods for the formation of silver nanoparticles. Geoprincy et al., “A Review on Green Synthesis of Silver Nanoparticles,” Asian J. Pharm. Clin. Res. 6:8-12 (2013); Hebbalalu et al., “Greener Techniques for the Synthesis of Silver Nanoparticles Using Plant Extracts, Enzymes, Bacteria, Biodegradable Polymers, and Microwaves,” ACS Sustainable Chem. Eng. 1:703-12 (2013); Park, Y., “A New Paradigm Shift for the Green Synthesis of Antibacterial Silver Nanoparticles Utilizing Plant Extracts,” Toxicol. Res. (Seoul, Repub. Korea) 30:169-78 (2014); Sharma et al., “Green Synthesis and Antimicrobial Potential of Silver Nanoparticles,” Int. J. Green Nanotechnol. 4:1-16 (2012); and Raveendran et al., “Completely ‘Green’ Synthesis and Stabilization of Metal Nanoparticles,” J. Am. Chem. Soc. 125:13940-1 (2003), all of which are hereby incorporated by reference in their entirety. In many cases the reactant is polysaccharide where contact with Ag⁺ is accompanied by metal ion reduction and the concomitant conversion of sugar aldehyde to carboxylic acid. In this two-electron process one equivalent of silver is bound to form a complex and the second may be deposited within the hydroxyl network of the polysaccharide.

The reaction of Ag⁺ with glucuronate groups of CDA gluc-NP was carried out by the addition of aqueous AgNO₃ to CDA-gluc-NP in water in the ratio was Ag⁺:CDA gluc-NP=6:1. In this system the silver redox reaction converts glucuronic acid to a monoprotic form of glucaric acid which has the capacity to bind up to two equivalents of the elemental silver that are produced in the reaction. After stirring for 15 minutes in a foil-shielded vial the brown solution was centrifuged using a Microsep™ tube to remove excess silver ions. UV-vis data from the solution revealed a surface plasmon resonance peak at 435 nm. This wavelength is relatively long and is perhaps due to pH effects or interparticle spacing between silver nanoparticles on/in the gluc-NP matrix. Rehman et al., “Synthesis and Optical Studies of Silver Nanoparticles (Ag NPs) and their Hybrids of Smart Polymer Microgel,” J. Chem. Soc. Pak. 35:717-25 (2013), which is hereby incorporated by reference in its entirety. An alternative view is that the long wavelength is a consequence of Mie resonance in very small particles. Peng et al., “Reversing the Size-Dependence of Surface Plasmon Resonances,” P. Natl. Acad. Sci. USA 107:14530-4 (2010), which is hereby incorporated by reference in its entirety. In either case the wavelength is in a range typical of Ag nanoparticles, which must be considered an adequate assessment at present in the analysis of the surface plasmon resonance of small Ag nanoparticles. Link et al., “Shape and Size Dependence of Radiative, Non-Radiative and Photothermal Properties of Gold Nanocrystals,” Int. Rev. Phys. Chem. 19:409-53 (2000) and Kreibig et al., “Optical-Absorption of Small Metallic Particles,” Surf Sci. 156:678-700 (1985), both of which are hereby incorporated by reference in their entirety.

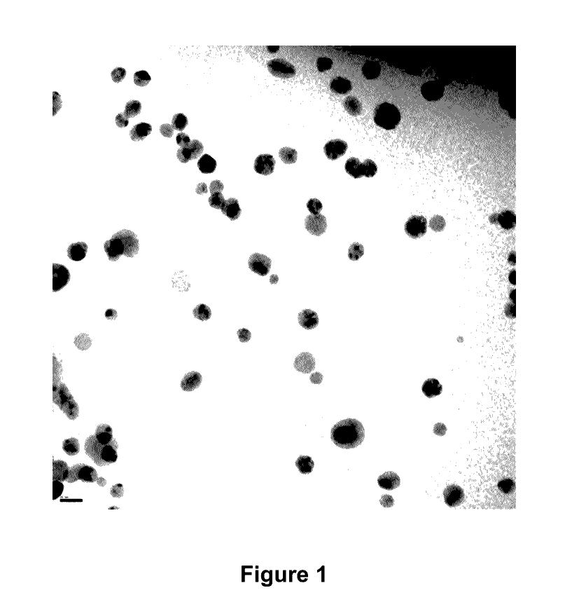

As shown in FIG. 1, electron microscopy revealed gluc-NP@Ag composite particles with an average diameter of 20 nm.

Based on size, which matches that of CDA gluc-NP, the reaction with silver produces composite nanoparticles, gluc-NP@Ag, where gluc=glucuronic_{y}-glucaric_{1-y} acid with monodeprotonation. Isolated high-density Ag nanoparticles were not observed in the TEM image. It appears that the distribution of silver approaches uniformity on or within the sugar nanoparticle matrix. The nature of silver in the gluc/water phase was discerned from powder diffraction data.

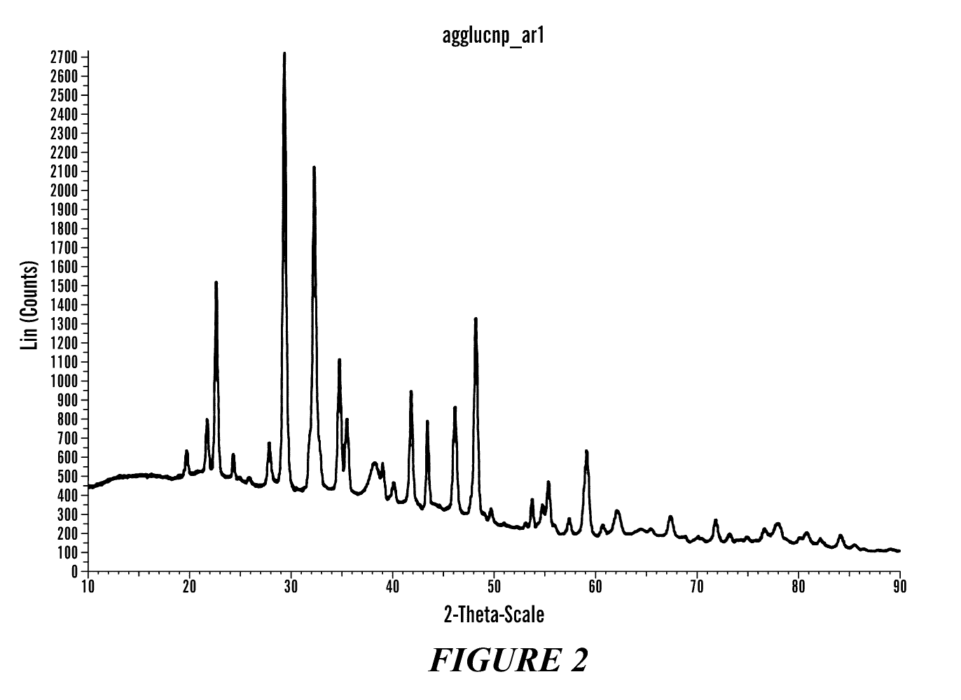

As shown in FIG. 2, the peak from the standard fcc phase of elemental Ag appears at 38.2 (111) while the expected peak at 44.5 (200) is absent probably because it is both broad and weak in the reflectance powder diffraction data of gluc-NP@Ag.

A second family of sharper more intense peaks belongs to two forms of orthorhombic AgNO₃ (Pbca and Imm2 space groups) signaling the propensity of the particle to retain salt even after a cycle of centrifugation and washing. Salt is likely sequestered in the particle matrix or core. The relatively small amount of nanoparticulate elemental Ag is a product of the polymer gluc that serves as a reducing sugar. In most polysaccharides, the reducing sugars are those that provide terminal hemiacetal units on the polymer chain. It is these units that provide the basis for the Tollens test. Similarly, in a hyperbranched network of gluc NP, hemiacetal forms of the sugar would be required to reduce Ag⁺ to its elemental form. The relatively weak peaks for Ag in the powder diffraction pattern are commensurate with the small number of reducing equivalents expected in the polymer. The ideal hyperbranched network that forms a closed capsule would have no terminal hemiacetal groups for the reduction of Ag⁺.

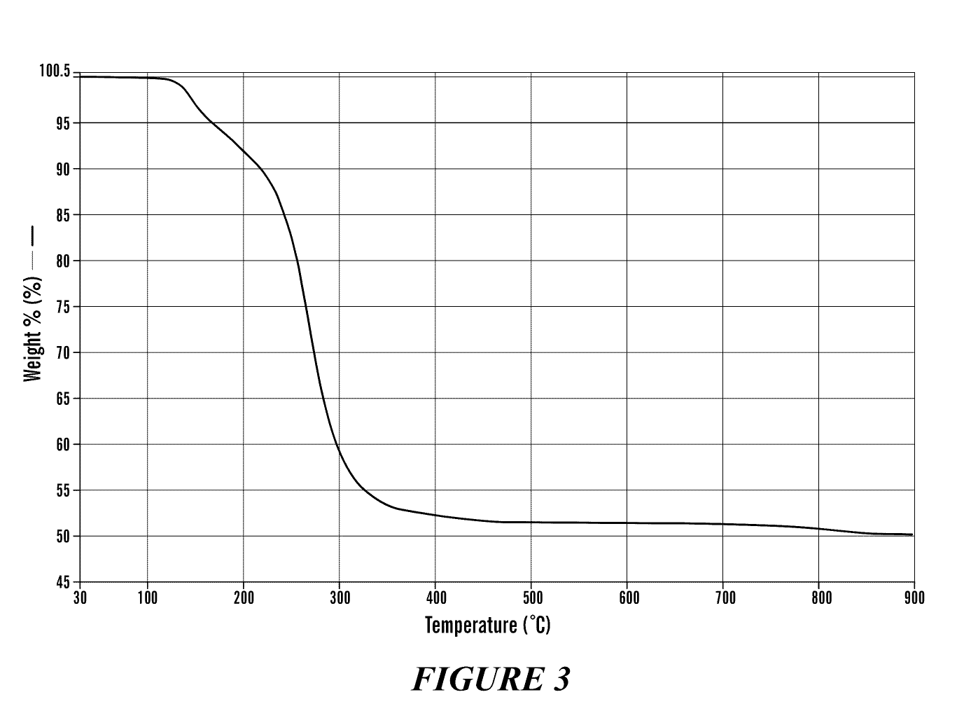

As illustrated in FIG. 3, thermogravimetric analysis showed a 17% mass decrease below 250° C. is attributed to water desorption. In the range 250° to 500° C. 37% of the mass decrease is attributable to the organic species. The mass remaining above 500° C. comprises 53% of the original sample mass and is assigned to purely elemental Ag. Aukrust et al., “Polymorphism of Gadolinium Diethylenetriaminepentaacetic Acid Bis(methylamide) (GdDTPA-BMA) and Dysprosium Diethylenetriaminepentaacetic Acid Bis(methylamide) (DyDTPA-BMA),” Acta. Chem. Scand. 51:18-26 (1997), which is hereby incorporated by reference in its entirety. The elemental Ag is derived from the binding of Ag⁺ to the particle with or without reduction, and the adsorption of AgNO₃ from the aqueous solution. The elemental Ag and AgNO₃ are detected by powder diffraction data. At high temperature any Ag⁺ in the sample is reduced to elemental Ag in the process of acquiring TGA data. Stern, K. H., “High Temperature Properties and Decomposition of Inorganic Salts, Part 3. Nitrates and Nitrites,” J. Phys. Chem. Ref. Data 1:747-72 (1972), which is hereby incorporated by reference in its entirety. Silver nitrate is likely absorbed by the particle from aqueous solution during the staining process. From the data above, 3.27 equivalents of Ag per gluc monomer were found.

### Example 14

### CDA-Gluc_{NP}/AgNP Superstructures

Yamamoto et al. showed that carboxylate groups control the nucleation and formation of silver nanoparticles from AgNO₃ in the refluxing triethylamine. Yamamoto et al., “Size-Controlled Synthesis of Monodispersed Silver Nanoparticles Capped by Long-Chain Alkyl Carboxylates From Silver Carboxylate and Tertiary Amine,” Langmuir 22:8581-6 (2006), which is hereby incorporated by reference in its entirety. In the absence of carboxylate groups a silver mirror forms on the surface of the reaction vessel. In the presence of myristic acid silver nanoparticles (Ag@myr) form that are uniform with a diameter of about 4 nm in diameters.

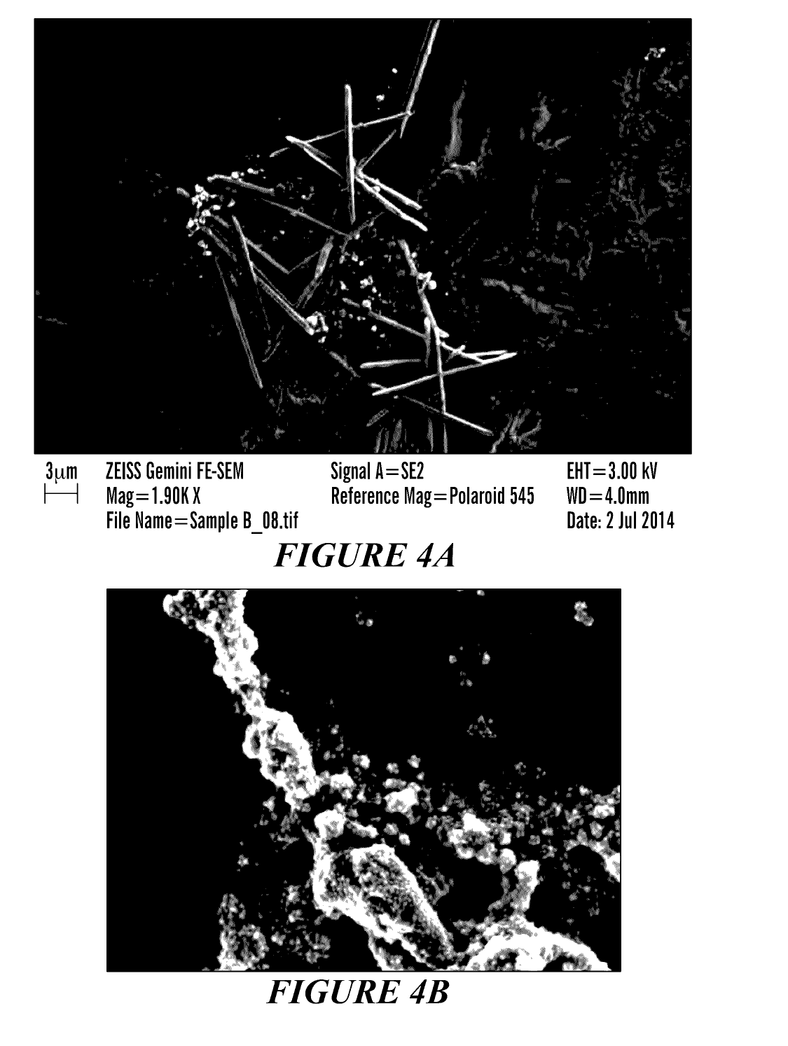

The simple process of mixing CDA glucNP with excess Ag@myr in CHCl₃ resulted in the formation of a dark brown solution with copious precipitate. SEM imaging of the precipitate revealed a range of superstructures that include grape clusters and microscale nanorods (see FIG. 4A). The insoluble products are formed by the reaction of CDA gluc-NP with Ag@myr, which likely produces a gluc-NP/Ag composite with soluble CDA myristate as a byproduct that is removed in the chloroform supernatant (see FIG. 4B).

The ready formation of easily isolable superstructures of gluc-NP/Ag@myr suggests that a similar controlled process could yield useful materials.

### Example 15

### Uniform Gluc-Rich Nanoparticles are Suitable for Incorporation of Ions and Chemical Derivatization of the Particle Surface

The aim was to prepare uniform glue-rich nanoparticles suitable for the incorporation of ions and chemical derivatization of the particle surface. The product gluc-NP was acquired by anionic polymerization through the reaction of glucuronate with epichlorohydrin in chloroform in a CDA reverse micelle. The polymerization is of the a_{z}+b₄ type, which results in two dimensional polymerization to form a capsule within, or surrounding, a water core. Typically, core crosslinked reverse micelles are derived from block copolymer precursors that require expertise in production and workup. The work here makes use of a commercially available and naturally occurring monomer, glucuronic acid. This approach should lend itself to new nanoparticles constructed of readily available precursors.

The structural data suggests that the polymeric nanoparticles derived from glue should assemble with the sugar carboxylate group oriented outward towards the cationic head group of the surfactant. It is demonstrated that the resulting particles are effective in the binding of silver ions and silver metal nanoparticles. Particles of this type should prove useful for catalysis and materials design.

### Example 16

### Manganese-Containing Nanoparticle in MRI Applications

A manganese-containing glucuronate-epichlorohyrin nanoparticle was designed as an MRI contrast agent. The synthesis of CDAglucNP with Mn(gluc)₂ is as described in Table 1 below.

TABLE 1
Synthesis of CDAglucNP with Mn(gluc)₂

| Equation | Mn(gluc)₂ + | CDAgluc + | CDAOH + | epichlorohydrin + | H₂O |
|---|---|---|---|---|---|
| Mole | 0.0002 mol | 0.0002 mol | 0.0012 mol | 0.0012 mol | 0.1666 mol |
| MW | 444 | 521 | 345 | 92.52 | 18 |
| Net mass | 0.0888 g | 0.1042 g | 0.414 | 0.111 | 0.2998 |

A mole of glucuronic acid needs 2 moles of epichlorohydrin to crosslink. From the equation, there were 0.0002 moles of Mn(gluc)₂ and 0.0002 moles of CDAgluc. Therefore, 0.0012 moles of epichlorohydrin were needed. CDAOH was added to balance the amount of epichlorohydrin. For the reaction, w=11.9. Thus, 0.1666 mol was calculated from all moles of the surfactants in the equation.

The procedure includes the following steps. First, CDAgluc and CDAOH were added to a flask to which 5 mL CHCl₃ was added. After CHCl₃ was added, Mn(gluc)₂ and water were added to the flask. Next, the solution was stirred under a reflux at 40° C. for 20 minutes. Epichlorohydrin was then added to the solution for polymerization and stirred under reflux for 18 hours. After stirring for 18 hours, CHCl₃ was removed by rotary evaporator. The product was then washed with ethyl ether. The crude product was dissolved in water and concentrated by Millipore centrifugation/filtration (3750 rpm/25° C./15 min). The concentrated solution was collected above the filter of the Millipore tube. The solution was lyophilized (freeze drying) to obtain the solid nanoparticulate product.

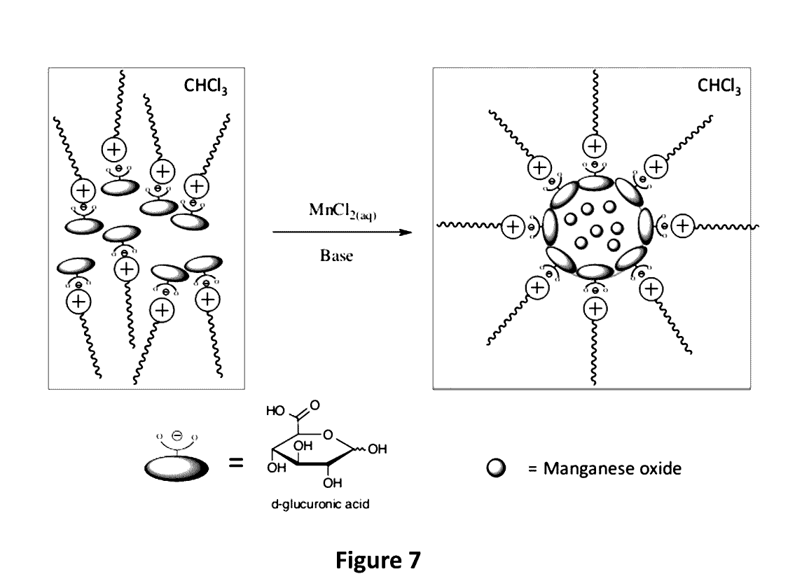

A synthetic overview for manganese oxide containing nanoparticles is illustrated in FIG. 7, where manganese oxide was formed from the reaction of MnCl₂ and water in the core of the CDA-gluc reverse micelle.

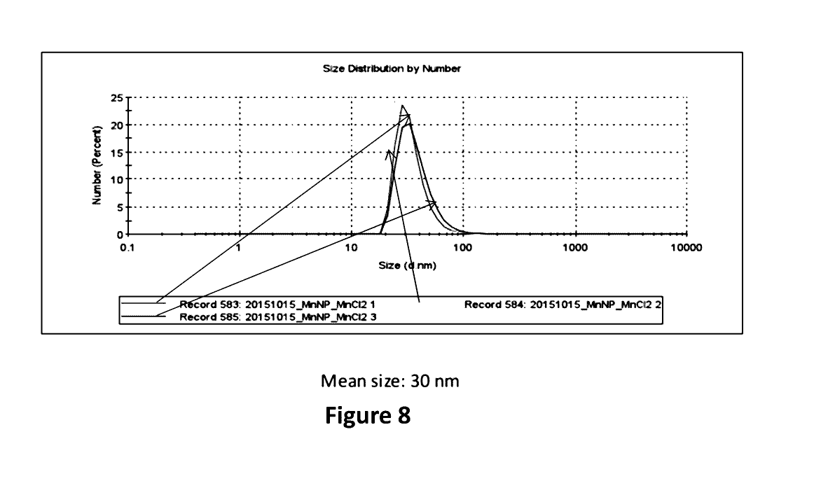

Dynamic Light Scattering Data (DLS) for the manganese oxide nanostructures are shown in FIG. 8. The three traces show slight shifts in the population of particles for each of three sets of scans.

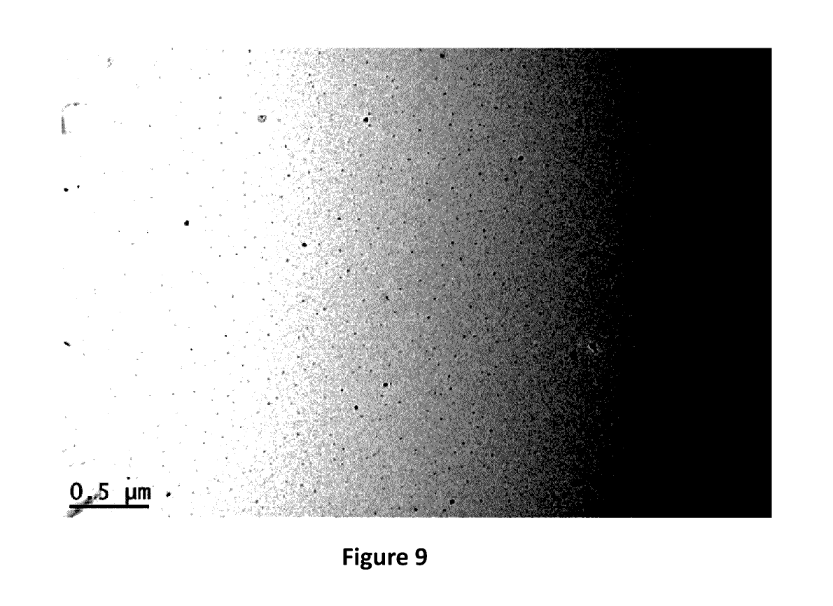

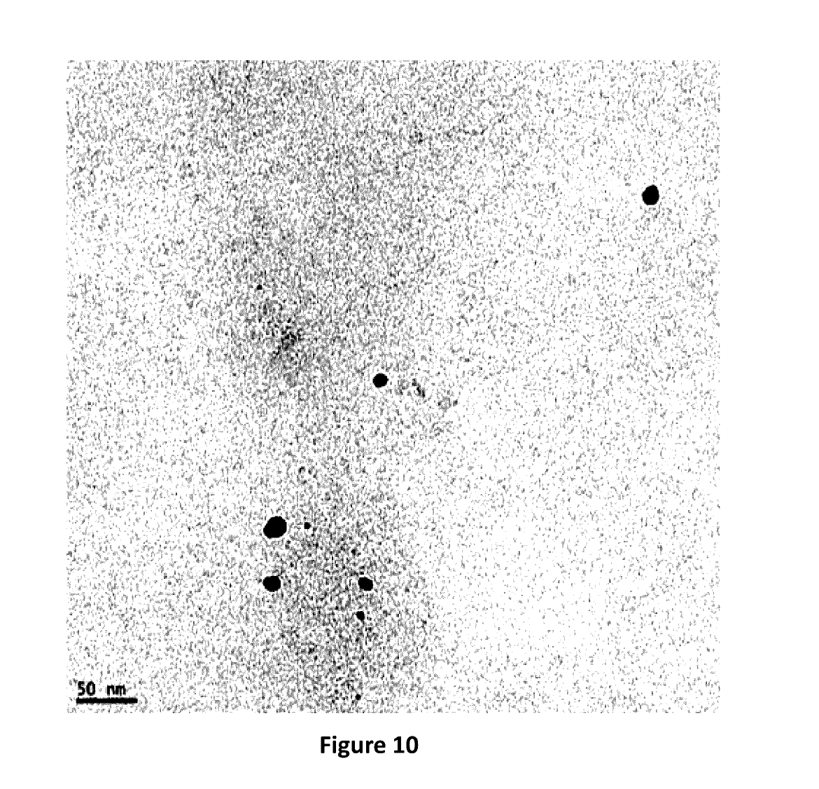

A low magnification TEM image for manganese oxide nanostructure is shown in FIG. 9 and a high magnification TEM image for manganese oxide nanostructure is shown in FIG. 10.

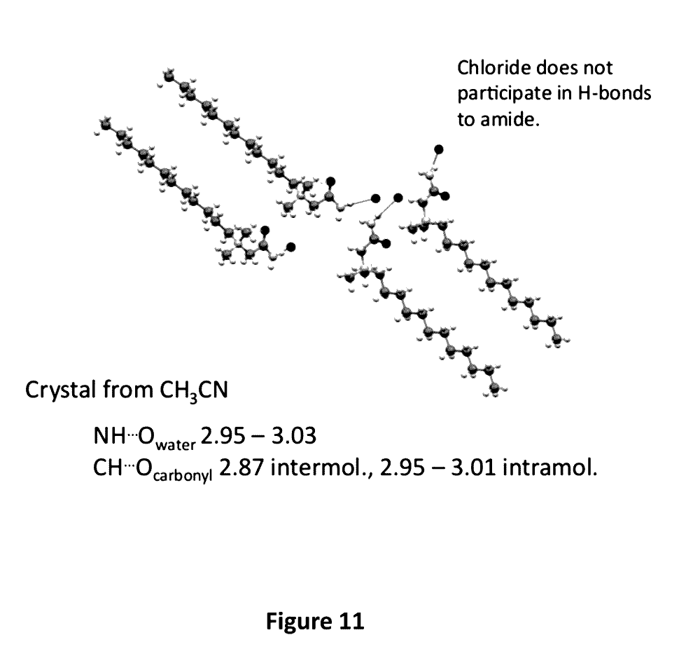

Details from the single crystal X-ray crystallographic structure of dodecyl dimethylammonium acetamido chloride (“DDA-Cl”) are shown in FIG. 11. As shown in FIG. 11, the surfactant headgroup is hydrogen bonded to water but not to chloride ions. The chloride ions are surrounded by water.

It is possible to exchange halide ions of octadecyl dimethylammonium (“ODA”), CDA, and dodecyl dimethylammonium acetamido (“DDA”) for other anions. If the anions are polar, they will engage in polar interactions with the amide headgroup of the surfactants. In the case of glucuronate, the polar interaction is expected to involve hydrogen bonding. The hydrogen bonding interaction is expected to stabilize reverse micelles.

TABLE 2
Solution Concentration of ODA, CDA, and DDA Halide Ions and Anions

| | Soln. conc. | | |
|---|---|---|---|
| | 15 mM | 4.5 mM | 2.25 mM |
| ODACl | — | 67.8 | 44.9 |
| CDACl | — | 52.89 | 41.22 |
| DDACl | — | 145 | 149.2 |
| ODAgluc | 31.57 | 39.13 | 3.157* |
| CDAgluc | 35.65 | 27.66 | 31.91 |
| DDAgluc | 282.9 | 422.4* | 183.1 |

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A nanoparticle comprising:
a shell formed from a first monomer having a first charge and a second monomer different than the first monomer, said first and second monomers being copolymerized, wherein said shell encapsulates a core region and is associated with a charged entity having a second charge of opposite sign to the first charge.

2. The nanoparticle of claim 1, wherein the charged entity is derived from a surfactant.

3. The nanoparticle of claim 2, wherein the charged entity is derived from a cationic surfactant selected from the group consisting of C₁₂-C₁₈ alkyl dimethylammonium acetamide, C₁₂-C₁₈ alkyl trimethylammonium, and mixtures thereof.

4. The nanoparticle of claim 1, wherein the charged entity is a charged substituent.

5. The nanoparticle of claim 4, wherein the charged substituent is a metal selected from the group consisting of silver, gold, copper, platinum, iron, manganese, cobalt, and mixtures thereof.

6. The nanoparticle of claim 1, wherein the first monomer and the second monomer are covalently linked.

7. The nanoparticle of claim 1, wherein the first charge is negative and the second charge is positive.

8. The nanoparticle of claim 1, wherein the first charge is positive and the second charge is negative.

9. (canceled)

10. The nanoparticle of claim 1, wherein the first monomer is a deprotonated sugar acid comprising three or more hydroxyl groups.

11. The nanoparticle of claim 1, wherein the first monomer is selected from the group consisting of carboxylic acids, amines, alcohols, thiols, aldehydes, ketones, ethers, esters, nitriles, imides, or any salt thereof.

12. (canceled)

13. The nanoparticle of claim 1, wherein the ratio of the first monomer to the charged entity is in the range of 3:1 to 1:3.

14. The nanoparticle of claim 1, wherein the nanoparticle is a hyperbranched polymeric nanoparticle.

15. The nanoparticle of claim 1, wherein the nanoparticle is a reticulated capsule.

16. The nanoparticle of claim 1, where the core region contains a core material selected from the group consisting of water, organic molecules, dye molecules, drugs, inorganic ions, organic ions, water soluble species, metals, and combinations thereof.

17.-23. (canceled)

24. A nanoparticle comprising:
a shell formed from a first monomer and a second monomer different from the first monomer, said first and second monomer being copolymerized, wherein said shell encapsulates a core region and has a neutral charge.

25.-26. (canceled)

27. A dispersion comprising:
the nanoparticle of claim 1 and
a medium selected from the group consisting of water, methanol, dimethyl sulfoxide, chloroform, methylene chloride, and mixtures thereof, wherein the nanoparticle is dispersed in the medium.

28. A method of making a nanoparticle, said method comprising:
providing a first monomer having a first charge;
providing a charged entity having a second charge of opposite sign to the first charge;
contacting the first monomer with the charged entity in an aqueous medium under conditions effective to form a complex where the first monomer is associated with the charged entity;
providing a second monomer; and
contacting the complex with the second monomer under conditions effective to form a shell of the first and second monomers which have been copolymerized, said shell encapsulating a core region containing an aqueous medium.

29.-38. (canceled)

39. A method of imaging comprising:
providing the nanoparticle of claim 1, wherein the nanoparticle encapsulates an imaging agent in the core region;
providing a subject to be imaged;
administering the nanoparticle to said subject; and
conducting an imaging procedure on said subject to which the nanoparticles have been administered.

40.-44. (canceled)

45. A method of delivering drugs, said method comprising:
providing the nanoparticle of claim 1 with a drug encapsulated in the core;
providing a subject to be treated; and
administering to a subject the nanoparticle under conditions effective to deliver drugs.

46. A method of delivering a high concentration of contrast enhancing and/or imaging agents, said method comprising:
providing the nanoparticle of claim 1 with a contrast enhancing and/or imaging agent encapsulated in the core;
providing a subject to be treated; and
administering to the subject the nanoparticle.
